(19) United States
(12) Patent Application Publication
(10) Pub. No.: US 2025/0295145 A1
(43) Pub. Date: Sep. 25, 2025

(54) NEUTRAL TASTE 1,3-BUTYLENE GLYCOL PRODUCTS AND COMPOSITIONS THEREOF

(71) Applicant: Genomatica, Inc., San Diego, CA (US)

(72) Inventors: Fasil A. Tadesse, San Diego, CA (US); Aakash Parikh, San Diego, CA (US)

(21) Appl. No.: 18/853,349

(22) PCT Filed: Apr. 10, 2023
(86) PCT No.: PCT/US2023/065595

§ 371 Date: Oct. 1, 2024

Related U.S. Application Data
(60) Provisional application No. 63/329,756, filed on Apr. 11, 2022.
(60) Provisional application No. 63/331,616, filed on Apr. 15, 2022.

(51) Int. Cl. A23L 2/52; A23L 27/20; C07C 31/20

(52) U.S. Cl. CPC A23L 2/52; A23L 27/2026; C07C 31/20

(57) ABSTRACT

The disclosure provides embodiments directed to a 1,3-butylene glycol (BG) product that has a neutral taste, a composition comprising the BG product described here, and methods of using the BG product and compositions comprising the BG products. BG products described here demonstrate exemplary qualities and characteristics of being natural, sustainable, and having a neutral taste for use in food products (including beverages). Such BG compositions of the description provide a BG product that does not mask or alter the taste of the food products. The BG products and compositions comprising such BG products as disclosed here provide advantageous properties while maintaining a neutral taste.

### RELATED APPLICATIONS

This application is a U.S. National Stage Application under 35 U.S.C. § 371 of International Patent Application No. PCT/US2023/065595, filed Apr. 10, 2023, which claims priority to U.S. Provisional Application Ser. No. 63/329,756, filed on Apr. 11, 2022; and U.S. Provisional Application Ser. No. 63/331,616, filed on Apr. 15, 2022; the disclosures of each of which are hereby incorporated by reference in their entireties.

### FIELD OF DISCLOSURE

The present disclosure is generally related to 1,3-butylene glycol (BG) products and compositions comprising such BG products, and methods and uses thereof, where BG products of the disclosure comprise an essentially neutral taste or flavor and/or neutral taste or flavor for use in food products, including beverages.

### BACKGROUND

The compound BG (also known and used interchangeably here as 1,3-butylene glycol; 1,3-BG; 1,3-butanediol; 1,3-BDO) is a four-carbon diol (C₄H₁₀O₂) traditionally produced from petroleum-derived acetylene through its hydration. The resulting acetaldehyde is then converted to 3-hydroxybutyraldehdye which is subsequently reduced to form a petroleum-derived 1,3-butylene glycol. However, this conventional petrochemical process begins with acetaldehyde, which is a mutagen and carcinogen. Moreover, petroleum-derived BG (petro-BG) is costly, lacks sustainability, has irritant properties, and can contain contaminants or compounds that result in products containing an offensive or undesirable odor and/or taste.

Therefore, there is a need to develop an alternative to petroleum-derived conventional materials, such as petro-BG, that can be used in cost-efficient compositions where the BG is safe, natural, and sustainable without offensive or undesirable tastes or flavors.

### SUMMARY

In accordance with the foregoing objectives and others, embodiments of the present disclosure satisfy the aforementioned needs and provide related advantages as well. The present disclosure provides in some aspects, a neutral taste or flavor 1,3-butylene glycol (BG) product, a composition comprising such a BG product, including food products (e.g., flavored beverages, flavored foods, high energy beverages, high energy foods, pharmaceuticals, nutraceuticals, supplements), and methods and uses of such BG products and compositions.

Some aspects of the disclosure are directed to a 1,3-butylene glycol (BG) product comprising a bioderived BG (bioBG), wherein the BG product does not comprise one or more compounds characterized by a flavor-active region as measured by ultra-high performance liquid chromatography-quadrupole time-of-flight mass spectrometry (UHPLC/QTOF-MS), wherein the flavor-active region elutes at a retention time selected from a group consisting of: ˜5.67 mins, ˜8.202 mins, ˜8.513 mins, ˜8.758 mins, ˜9.002 mins, ˜9.435 mins, and any combinations thereof, wherein the bioBG elutes at a retention time of ˜1.005 mins.

In other aspects, a 1,3-butylene glycol (BG) product comprising a bioderived BG (bioBG) is provided, wherein the BG product does not comprise one or more compounds characterized by a flavor-active region as measured by ultra-high performance liquid chromatography-quadrupole time-of-flight mass spectrometry (UHPLC/QTOF-MS), wherein the flavor-active region comprises a relative retention time selected from a group consisting of: ˜5.614 mins, ˜8.121 mins, ˜8.429 mins, ˜8.671 mins, ˜8.913 mins, ˜9.341 mins, and any combinations thereof, where the bioBG relative retention time is 1 min.

Additional aspects provide a 1,3-butylene glycol (BG) product comprising a bioderived BG (bioBG), wherein the BG product does not comprise one or more compounds characterized by a flavor-active region as measured by ultra-high performance liquid chromatography-tandem mass spectrometry (UHPLC-MS/MS) at a collision energy of 10 V, wherein the flavor-active region elutes at a retention time selected from the group consisting of: ˜2.658 mins, ˜4.801 mins, ˜5.493 mins, ˜5.683 mins, ˜6.047 mins, ˜6.237 mins, ˜7.978 mins, ˜8.343 mins, ˜8.817 mins, ˜9.275 mins, ˜13.229 mins, and any combinations thereof, wherein the BG product comprises a bioBG elutes at a retention time of ˜0.996 min.

In some aspects, a 1,3-butylene glycol (BG) product comprising a bioderived BG (bioBG) is provided, wherein the BG product does not comprise one or more compounds characterized by a flavor-active region as measured by ultra-high performance liquid chromatography-tandem mass spectrometry (UHPLC-MS/MS) at a collision energy of 10 V, wherein the flavor-active region comprises a relative retention time selected from the group consisting of: ˜2.679 mins, ˜4.840 mins, ˜5.537 mins, ˜5.729 mins, ˜6.096 mins, ˜6.287 mins, ˜8.042 mins, ˜8.410 mins, ˜8.888 mins, ˜9.350 mins, ˜13.335 mins, where the relative retention time of BG is 1 min.

Other aspects provide a 1,3-butylene glycol (BG) product comprising a bioderived BG (bioBG), wherein the BG product does not comprise one or more compounds comprising a mass spectrum as characterized by ultra-high performance liquid chromatography-tandem mass spectrometry (UHPLC-MS/MS) at a collision energy of 10 V, wherein the mass spectrum is selected from the group consisting of:
(a) 191.0737 m/z, 172.977 m/z, 141.9585 m/z, 87.0804 m/z, and 69.0700 m/z;
(b) 127.0774 m/z, 109.0650 m/z, and 81.0699 m/z;
(c) 204.1591 m/z, 159.1012 m/z, 141.9584 m/z, 89.0593 m/z, and 60.0803 m/z;
(d) 143.1070 m/z, 103.0756 m/z, 89.0599 m/z, and 71.0493 m/z;
(e) 206.1753 m/z, 143.1070 m/z, 86.0966 m/z, and 60.0807 m/z;
(f) 206.1757 m/z, 161.1178 m/z, 143.1070 m/z, 89.0601 m/z, and 71.0494 m/z;
(g) 103.0751 m/z, 89.0595 m/z, 71.0489 m/z, and 60.0804 m/z;
(h) 310.2595 m/z, 296.2439 m/z, 141.9597 m/z, 100.1129 m/z, 86.0973 m/z, and 60.0815 m/z;
(i) 310.2585 m/z, 296.2432 m/z, 161.1171 m/z, 86.0962 m/z, and 60.0805 m/z; 0) 167.1067 m/z, 141.9587 m/z, 86.0964 m/z, and 60.0806 m/z;
(k) 199.1331 m/z, 141.9586 m/z, 100.1120 m/z, and 86.0964 m/z; and any combinations thereof, wherein the BG product comprises a bioBG mass spectrum of: 91.0756 m/z, 73.0648 m/z, and 55.0541 m/z. Further objects of the BG product are directed to the BG product comprising BG (e.g., bioBG), wherein the BG is selected from the group consisting of: (R)-1,3-BG; (S)-1,3-BG; and a mixture of (R)-1,3-BG and (S)-1,3-BG. Additional objects provide the BG product comprising BG (e.g., bioBG), wherein the BG comprises (R)-1,3-BG. In some objects, the BG product comprising a BG of the disclosure (e.g., bioBG), wherein the BG comprises (S)-1,3-BG. Additional objects provide the BG product comprising a BG (e.g., bioBG) of the disclosure, wherein the BG comprises a mixture of (R)-1,3-BG and (S)-1,3-BG. In other objects, the BG product of the disclosure comprises a neutral taste. Further objects provide the BG product comprising a BG, such as a neutral taste bioBG, wherein the BG product does not comprise a biting, irritating bitterness.

In some aspects, a composition, comprising: the 1,3-butylene glycol (BG) product described here, and a physiologically acceptable vehicle is provided. Additional objects provide the composition of the disclosure comprising a BG (e.g., bioBG) product, wherein the BG product comprises a neutral taste. In other objects, the composition of the disclosure comprising a BG (e.g., bioBG) product described here, wherein the BG product comprises a property selected from the group consisting of or wherein the BG product is: a solvent, a hypoglycemic agent, a fungicide, a bactericide, a preservative, a food additive, a calorie source, and combinations thereof. Further objects provide the composition of the disclosure, wherein the BG product does not comprise compounds characterized by peaks as measured by ultra-high performance liquid chromatography-quadrupole time-of-flight mass spectrometry (UHPLC/QTOF-MS), wherein the peaks elute at a retention time of: ˜5.67 mins, ˜8.202 mins, ˜8.513 mins, ˜8.758 mins, ˜9.002 mins, and ˜9.435 mins. In some objects, the composition described here is a food product, including beverages. Other objects provided here include the composition selected from the group consisting of: flavored beverages, flavored foods, high energy beverages, high energy foods, pharmaceuticals, nutraceuticals, supplements, and the like. In additional objects, the composition is a high energy beverage. In additional objects, the composition comprises a BG product described here, wherein the BG product comprises a neutral taste bioderived (R)-1,3-BG in an amount about 1 mg/oz to about 200 mg/oz. Further objects provide the composition comprising a BG product described here, wherein the BG product does not comprise a biting, irritating bitterness.

### BRIEF DESCRIPTION OF FIGURES

FIG. 1 shows an exemplary block flow diagram for obtaining neutral taste bioderived 1,3-butylene glycol (bioBG) using a caustic reaction followed by multi-step distillation to produce bioBG Product 1 (DSP-555-2). Values are normalized to 100 kg of feed.

FIG. 2 shows chromatograms (upper panel) obtained after a first-dimension fractionation of the off-flavor sample (DSP-650-3) using preparative HPLC/MS with the RP-C18 column. The lower panel illustrates the average bitter taste intensity of subfractions (F1-F6) in water rated by 6 trained sensory panelists on a 5-point scale from: not present (0) to strong (4).

FIG. 3 shows the average bitter taste intensity of off-flavor (DSP-650-3) SPE subfractions (F1-F7) in water as rated by 6 trained panelists on a 5-point scale from: not present (0) to strong (4). SPE and sensory evaluation were performed in duplicates.

FIG. 4A shows a chromatogram of subfraction F1 obtained from prep-HPLC fractionation of the off-flavor sample (DSP-650-3). FIG. 4B shows a chromatogram of subfraction F7 obtained from preparative SPE fractionation. Both chromatograms were analyzed by UHPLC/MS instrument.

FIG. 5 shows the average bitter taste intensity of subfraction F1 obtained from SPE fractionation (column 1); standard solvent of 1,3-butylene glycol (column 2); neutral taste bioBG sample (DSP-4 or DSP-650-4; column 3); and off-flavor sample (DSP-3 or DSP-650-3; column 4) as rated by 6 trained panelists on a 5-point scale from: not present (0) to strong (4). Sensory evaluation was performed in duplicates. All samples were evaluated at a concentration of 4% (v v).

FIG. 6 shows a comparative chromatogram of off-flavor (DSP-650-3) and neutral taste bioBG (DSP-650-4) samples by UHPLC/MS. Internal standard (ISTD).

FIG. 7 shows overlaid chromatograms of 100-fold dilution solutions of neutral taste bioBG samples, 1,3-butylene glycol standard solution, and methanol blank.

FIG. 8 shows a zoomed-in portion of the overlaid chromatograms from FIG. 7 of a peak present in the neutral taste bioBG samples and not observed in the 1,3-butylene glycol standard solution or methanol blank.

FIG. 9 shows a NIST library match of the peak present in the neutral taste bioBG samples and not observed in the 1,3-butylene glycol standard solution or methanol blank of FIG. 8.

FIG. 10 shows overlaid chromatograms of headspace of neutral taste bioBG samples and 1,3-butylene glycol standard (1,3-butylene glycol method).

FIG. 11 shows a zoomed-in portion of the overlaid chromatograms from FIG. 10 of a peak present in the neutral taste bioBG samples identified as toluene.

FIG. 12 shows a NIST library match of the peak present in the neutral taste bioBG samples of FIGS. 10-11 identified as toluene.

FIG. 13 shows overlaid chromatograms of headspace of neutral taste bioBG samples, 1,3-butylene glycol standard, and toluene standard (volatiles method).

FIG. 14 shows a zoomed-in portion of the overlaid chromatograms from FIG. 13 of a peak present in the neutral taste bioBG samples and toluene standard and identified as toluene.

FIG. 15 shows a NIST library match of the peak identified as toluene in the neutral taste bioBG samples of FIGS. 13-14.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component (e.g., fragrance ingredient) relative to the entire weight of the composition, unless otherwise defined.

Demand for neutral taste and/or neutral taste additives in cosmetics or personal care products and/or nutraceutical or food compositions, respectively, is desirable.

### Butylene Glycol (BG) Product

In some embodiments, a purified 1,3-butylene glycol (synonymous and used interchangeably with BG; 1,3-BG; 1,3-butanediol; 1,3-BDO; butane-1,3-diol; etc.) can be present in a product (BG product) of the disclosure. CAS No. 107-88-0 is assigned to BG having a chemical formula of C₄H₁₀O₂. There are various methods for producing 1,3-butylene glycol. For example, U.S. Pat. No. 6,376,725, which is incorporated herein by reference in its entirety, describes methods of liquid phase hydrogenation of acetaldol (3-hydroxybutanal or aldol) with a Raney nickel catalyst resulting in the production of 1,3-butylene glycol. Another method of producing 1,3-butylene glycol is described as having three steps: aldol condensation of acetaldehyde to aldoxane, then decomposition of the aldoxane resulting in paraldol, which is then hydrogenated to finally produce 1,3-butylene glycol (as described in, e.g., U.S. Pat. Nos. 5,345,004; 5,583,270, which are incorporated herein by reference in their entirety).

Some embodiments of the disclosure are directed to a bioderived BG that has been purified and present in a BG product described here to form a bioderived BG product. BG can be a bioderived 1,3-butylene glycol (bioBG), which can include any type or form of BG, including but not limited to (R)-1,3-BG; (S)-1,3-BG; mixtures of (R)-1,3-BG and (S)-1,3-BG; or any combinations or mixtures of the aforementioned. In some examples, BG is a sustainable, natural bioderived 1,3-BG (bioBG), which can be produced by non-naturally occurring microorganisms that express genes encoding enzymes that catalyze bioBG production. The methods and compositions for producing bioBG through culturing of non-naturally occurring microbial organisms are described in, for example, WO 2010/127319 (e.g., Examples I-III), WO 2011/071682 (e.g., Examples I, III), and WO 2012/177619 (e.g., Examples I-IV, VIII), which disclose methods, vectors, strains, and culturing materials and conditions for producing bioderived 1,3-BG; and WO 2018/183628 (e.g., Example 1) and WO 2018/183664, which specifically disclose engineered organisms and enzymes producing bioderived 1,3-BG, as well as characterization of bioderived 1,3-BG, each of which is incorporated by reference in its entirety.

In some embodiments, the BG product or bioBG product of the disclosure can be purified and obtained by any one of the commonly known and used methods, including but not limited to, distillation, solvent extraction, and enfleurage (e.g., steam distillation, hydrodistillation, cohobation, molecular distillation, cold pressed extraction, and sieving). For example, solvent extraction can be performed by mixing a sample comprising 1,3-BG with a mixture of water and dichloromethane (DCM) (e.g., 1:1:1; 1:2:2). The mixture can be vigorously mixed and then the DCM layer collected. The DCM layer can then be concentrated to a volume (e.g., 200 μL) under nitrogen gas. In some embodiments, the concentrated extract can be measured by gas chromatography/mass spectrometry/olfactory (GC/MS/O) by injecting, for example, 1 μL into the device. The extract can be analyzed by a panel of trained sensory experts to assess differentiating aroma attributes between the concentrated extract and control samples. The chemical profile of the extract can be determined and analyzed by mass spectrometry. For example, neutral taste bioBG samples can comprise bioderived 1,3-BG in a weight percent of about 99 weight (wt) % or more (e.g., 99.1, 99.2, 99.3, 99.4, 99.5, 99.6, 99.7, 99.8, 99.9, 100). Some non-limiting samples can comprise 10 wt % 1,3-BG in deionized water, less than 10 wt % 1,3-BG in water that can contain other impurities, or 90 wt % 1,3-BG and other impurities.

As used herein, the term “non-naturally occurring” when used in reference to a microbial organism or microorganism of the disclosure is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, but are not limited to, coding regions and functional fragments thereof, for heterologous, homologous, or both heterologous and homologous polypeptides for the referenced species. Additional modifications include, for example, non-coding regulatory regions in which the modifications alter expression of a gene or operon. Exemplary metabolic polypeptides include enzymes or proteins within a 1,3-butanediol biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof.

These microorganisms can be supplied a source of carbon via any carbohydrate source. Such sources include, but are not limited to, sugars (e.g., hexoses: glucose, fructose, galactose, mannose; pentoses: xylose, arabinose) and starches. Plant biomass, an exemplary carbohydrate source, is a type of renewable organic material from plants that can be converted into chemicals, biofuels, and other materials. Non-limiting carbohydrate sources include renewable plant biomasses and feedstocks, such as but not limited to, cellulosic biomass, hemicellulosic biomass, and lignin feedstocks or portions of feedstocks.

As used herein, the term “bioderived” means derived from or synthesized by a biological organism and can be considered a renewable resource since a bioderived product can be generated by a biological organism. Such a biological organism, in particular, the microbial organisms of the disclosure described here, can utilize feedstock or biomass, such as, sugars or carbohydrates obtained from an agricultural, plant, bacterial, or animal source. Alternatively, the biological organism can utilize atmospheric carbon. As used here, the term “biobased” means a product as described here that is composed of, in whole or in part, a bioderived compound of the disclosure. A biobased or bioderived product of the disclosure (e.g., bioderived 1,3-butylene glycol or bioBG) is not, and is in contrast to, a “petroleum-derived product,” where such a petroleum-derived product is derived from or synthesized from petroleum or a petrochemical feedstock. A bioderived 1,3-butylene glycol used here can be a natural product as measured by the International Organization for Standardization (ISO). ISO 16128-1:2016 provides definitions for organic and natural cosmetic ingredients, and ISO 16128-2:2017 provides guidelines and criteria for determining natural, organic, or natural and organic origins of cosmetic ingredients and products.

### Process of Producing Neutral Taste BG Product

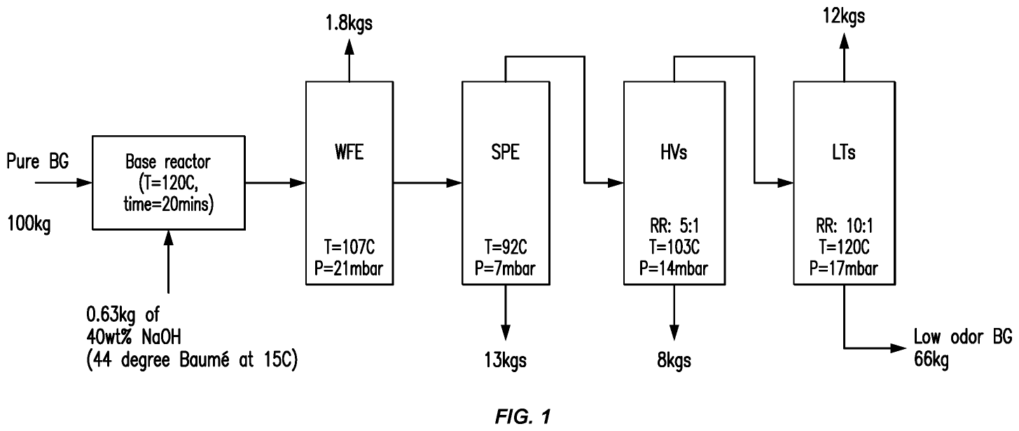

A product can be treated to remove any undesirable flavors using, for example, a distillation process. See, EXAMPLE 1; FIG. 1. Briefly, the crude bioBG containing undesirable flavors can be treated using a multi-step distillation process after reacting it with a caustic solution, such as a sodium hydroxide (NaOH) solution of about 40 wt % to about 50 wt %, in order to obtain a final concentration of, for example, 0.25 wt % NaOH (FIG. 1). The mixture of crude bioBG and caustic solution (e.g., NaOH, NaBH₄) can be reacted in a caustic reactor or tank vessel resulting in a reaction product. The reaction product can then be fed into one or multiple distillation columns to ultimately produce a final purified BG, low flavor BG, or a neutral taste BG (interchangeably used). The distillation process can comprise multiple lights removal and heavies removal. The lights waste can also be collected and re-processed. The final neutral taste BG can be used in a neutral taste BG product or composition comprising a neutral taste BG product as described here, such as food products for consumption by a subject or consumer.

An exemplary conventional purification method can include a method in which a reaction mixture of, for example, 1,3-butylene glycol (e.g., a bioderived 1,3-BG provided herein) produced by liquid phase hydrogen reduction of acetaldol, is subjected to removal of alcohols, removal of water, removal of salts, and removal of high-boiling materials. Then in a low-boiling material removal distillation tower (product distillation tower), low-boiling materials can be distilled off from the top of the tower and 1,3-butylene glycol (e.g., a neutral taste bioderived BG) can be obtained as a product from the tower bottom. The addition of a base to bioderived 1,3-butylene glycol having a low content of high-boiling materials and heat-treatment of the mixture effectively decreases undesirable flavor-causing materials and gives rise to a neutral taste bioderived 1,3-butylene glycol or a 1,3-butylene glycol having low to no taste or flavor which shows less change with time. The base to be added can include but is not limited to: an alkali metal compound, sodium hydroxide, potassium hydroxide, sodium (bi)carbonate, sodium hydroxide, potassium hydroxide or mixtures thereof. The base can be added in the form of solids as it is or can be added as an aqueous solution for ease of operation and for promoting contact with the target solution. Suitable base reactor and distillation reaction temperatures and conditions are provided in FIG. 1. In some embodiments, a distillation reaction temperature ranges from at least about 90 degrees Celsius (° C.) up to about 140° C. In some embodiments, the reaction retention time is at least about 5 minutes up to about 120 minutes. In some embodiments, the reaction retention time is at least about 10 minutes up to about 30 minutes.

From the top of the tower, bioderived 1,3-butylene glycol containing low-boiling materials is distilled off and is charged to the next product distillation tower. The product distillation tower may be a porous plate tower, a foamed bell tower, etc. The product distillation tower can be a filled tower having a low pressure loss, filled with Sulzer Packing, Melapack™ (both are trade names for products by Sumitomo Heavy Industries, Ltd.), etc., which is more suitable than other fillers. Without being bound by theory, the suitability is because 1,3-butylene glycol is thermally decomposed at 200° C. or higher which adversely affects odor and/or taste (See, e.g., U.S. Pat. No. 6,376,725 B1, the contents of which is incorporated herein by reference in its entirety). Accordingly, distillation temperature should be lowered as low as possible. When thermal hysteresis (retention time) to 1,3-butylene glycol is long, similarly influenced. Therefore, the reboiler to be adopted is suitably a thin film evaporator, such as a natural flow-down type thin film evaporator or a forced stirring type thin film evaporator.

The product distillation can depend on the concentration of low-boiling material in the charge stock solution, but when the concentration of low-boiling material in the charge stock solution is 5% or less, it can be one having a theoretical number of daylights of about 10 to 20 (trays). The charge stock solution is fed at a position from the top of the tower to a height 20 to 70% of the height of the tower. At this time, distillation is performed under reduced pressure of 100 torr (133.322 millibars (mbar)) or less, preferably 5 torr to 20 torr (6.66612 mbar-26.6645 mbar), at the top of the tower. For the undesirable flavors or tastes of a crude bioderived 1,3-butylene glycol to be removed, it is preferred to use a lower distillation temperature. The lower the pressure is, the more suitable. It is desirable to run distillation at a reflux ratio (RR) of at least about 0.5 up to about 15.

The charging into the product distillation tower is carried out by feeding the liquid obtained by concentrating the top of the tower vapor of the dealkalization tower in the condenser. To decrease the calorie for heating the product distillation tower, the top of the tower vapor from the dealkalization tower may be directly fed to the production tower. Bioderived 1,3-butylene glycol product can be obtained from the tower bottom of the product distillation tower. Suitable distillation conditions for generating a low taste or neutral taste bioderived BG are further described in Example 2.

Alternative distillation protocols known in the art can be used. By way of example only, the treated liquid from an alkali reactor can be fed first into the low-boiling material removal distillation tower where low-boiling materials are distilled off, subsequently the bioderived 1,3-butlene glycol extracted from the gas phase portion in the recover trays or from the tower bottom is distilled or evaporated to remove the base and the resulting high-boiling materials as distillation residues and 1,3-butylene glycol is recovered from the top of the tower or in the midway of concentration trays. The distillate of the above low-boiling materials can further be fed to an additional distillation tower where 1,3-butylene glycol can be distilled off to remove high-boiling materials. Also, a portion of the 1,3-butylene glycol after removal of the above low-boiling materials can be recycled to the dealkalization tower. Alternatively, low-boiling materials containing 1,3-butylene glycol can be recycled to the alkali reactor.

A caustic process can occur in an alkali reactor (see, e.g., U.S. Pat. No. 6,376,725, which is incorporated herein by reference in its entirety) or by simply adding a caustic solution, such as, caustic soda, caustic potash, sodium borohydride, and potassium borohydride during deboiling distillation (see, e.g., JP 3369707 B2, which is incorporated herein by reference in its entirety). Accordingly, some embodiments of the disclosure provide a method of obtaining a neutral taste BG comprising one or more (e.g., 2, 3, 4) distillations for use following any caustic (e.g., alkali) process with or without an alkali reactor.

The final purified BG can be used in a neutral taste BG product or composition comprising a neutral taste bioBG product as described here. A neutral taste, as used here, can be or can essentially be an undetectable taste, where a neutral taste can be below a taste threshold. In some embodiments, the neutral taste/flavor bioBG product can comprise, consist essentially of, consist of, or minimally comprise one or more off-flavor compounds which can be responsible for a flavor region or a flavor-active region described here, where the one or more compounds can be present in an amount below its respective taste or flavor threshold. In some uses, a composition can have a desired flavoring additive that is unaffected by the neutral taste components or ingredients described here.

### Neutral Taste BG Product

Embodiments of the disclosure are directed to a 1,3-butylene glycol (1,3-BG; BG) product comprising BG, where the BG product is removed of or essentially removed of any or all contaminants or compounds, which can have an undesirable or offensive taste. Some embodiments are directed to a BG product of the disclosure, where the BG product comprises a bioderived BG (bioBG) and comprises a neutral taste. In some embodiments, any of the BG products of the disclosure can comprise a neutral taste or flavor bioBG, where the neutral taste bioBG and/or one or more compounds present therein, can be present in an amount below its taste or flavor threshold. In some embodiments, any of the BG products of the disclosure can comprise a neutral taste bioBG, where the neutral taste bioBG is present in an amount below its taste or flavor threshold. The BG product can be a liquid or a viscous liquid. In some embodiments, the BG product can be an isolated or a purified liquid.

As used herein, the term “flavor-active region” is intended to mean an identifiable region within a fractionated sample of 1,3-butylene glycol (BG). The fractionation method can be any method known in the art that separates components based on, for example, chemical and/or physical properties. The fractionation method exemplified in this disclosure is High-Performance Liquid Chromatography (HPLC) and solid-phase extraction (SPE) as exemplified in EXAMPLE 2. The taste for the various BG fractions is determined by a sensory taste panel composed of trained individuals. See, e.g., EXAMPLES 2-3. In some embodiments, toluene can influence taste, or can be a contributor to “off-flavor”, which, as used here, can be a taint in food products caused by the presence of compounds that results in an undesirable taste or flavor (e.g., bitter). In additional embodiments, any of the one or more compounds and/or flavor-active regions associated with compounds described here can contribute to “off-flavor”. Some embodiments can provide such compounds and/or flavor-active regions in an amount or concentration below a taste/flavor threshold.

As used herein, the term “perception threshold” is intended to mean a specific level below which a compound is not detectable, and above which it can be detected by persons with average sensory acuity. For example, an “odor threshold” is intended to mean the point at which 50% of a panel of sensory odor panelists cannot, for example, smell an odor and which corresponds to one (1) odor unit per cubic meter (1 o.u./m³). For example, a “taste or flavor threshold” is intended to mean the point at which 50% of the sensory taste panelists cannot, for example, taste or perceive a taste or flavor of a substance. For example, a taste or flavor above the perception threshold or taste/flavor threshold can be considered an offensive or undesirable taste or flavor as determined by the sensory taste panelists. Exemplary taste or flavor thresholds can include a concentration for citric acid about 2 mM; for salt (NaCl) about 10 mM; and for sucrose about 20 mM. However, for potentially dangerous substances, such as those from bitter-tasting plant compounds, can have a taste or flavor threshold at much lower concentrations. For example, the threshold concentration for quinine is 0.008 mM and for strychnine is 0.0001 mM (see, e.g., Neuroscience (2^{nd} edition). Purves et al. editors. Sunderland (MA): Sinauer Associates, 2001, which is incorporated herein by reference in its entirety and particularly with respect to taste perceptions).

As used herein, the term “neutral taste” or “neutral flavor”, used interchangeably here, refers to a non-offensive taste or flavor to the gustatory system as determined by a subject, machine, sensory taste panelist, or any combination thereof. A “neutral taste BG”, “neutral taste bioderived BG”, a “neutral taste bioBG”, a “neutral flavor BG”, a “neutral flavor bioderived BG”, a “neutral flavor bioBG”, all of which are used interchangeably here, and grammatical equivalents thereof, refer to a bioderived 1,3 butylene glycol that does not have or minimally comprises an offensive taste or flavor. The terms can also refer to a bioderived 1,3-butylene glycol composition that has a flavor-active region or taste-active region that is below the perception threshold for the taste associated with the flavor- or taste-active region.

As used herein, the term “low taste” or “low flavor”, used interchangeably herein, and its grammatical equivalents, refers to a non-offensive taste or flavor to the gustatory system that is below the perception threshold as determined by a subject, machine, or sensory taste panelist. The terms “low taste bioderived butylene glycol,” “low taste bioBG,” “neutral taste BG,” “neutral taste bioBG”, “low flavor bioderived butylene glycol,” “low flavor bioBG,” or “neutral flavor BG,” or “neutral flavor bioBG”, used interchangeably herein, refer to a bioderived 1,3-butylene glycol composition that has an taste- or flavor-active region that is below the perception threshold for the taste or flavor associated with the corresponding taste- or flavor-active region based on, for example, a UHPLC/MS and MS/MS chromatograph. For example, a neutral flavor bioBG composition of the disclosure can comprise a flavor-active region that does not comprise, does not consist essentially of, does not consist of, or minimally comprises a relative retention time selected from the group consisting of ˜5.614 mins, ˜8.121 mins, ˜8.429 mins, ˜8.671 mins, ˜8.913 mins, ˜9.341 mins, and any combinations thereof, that is below the perception threshold for that taste or flavor (e.g., bitterness) and/or would not be perceived by a panel of trained professionals or a machine to have a bitterness taste. In some cases, a neutral taste or neutral flavor bioBG product is produced by removing one or more chemical compounds as described here from an unpurified, less purified, or crude bioderived BG product. In some embodiments, such one or more compounds can be in an amount or concentration below its respective taste or flavor threshold in a neutral taste or neutral flavor bioBG.

As used herein, the term “flavoring substance” or “flavoring additive” is intended to mean a material that provides or enhances a particular taste or smell. The material can include a solid, powder, a liquid, or a gas, for example. The flavoring substance can be a natural or artificial flavor. In some instances, the neutral taste BG product described herein can be a flavoring substance. In some embodiments, a chemical or flavoring substance can be added to a neutral taste bioBG composition provided herein to produce a flavored bioBG composition.

As used herein, the term “comprising” is intended to mean that the BG products, compositions comprising such BG products, and methods of using the BG products or compositions thereof as described here include the recited elements, but do not exclude others. “Consisting essentially of” when used to define the BG products, compositions, and methods, shall limit the scope of a claim to the specified materials or steps and those that do not materially affect the basic and novel characteristics of the claimed invention, or affect taste or flavor. Thus, a BG product or composition consisting essentially of the elements as defined herein could exclude trace contaminants from the isolation and purification method. “Consisting of” shall exclude any compound, element, or step not specified in the claim. “Minimally comprises” when used here to define the BG products and compositions disclosed, refers to one or more compounds that can contribute to off-flavor or off-taste in an amount or concentration that is, for example, below its taste or flavor threshold.

Some embodiments of the disclosure are directed to a BG product comprising a bioderived BG (bioBG), where the BG product of the disclosure does not comprise, does not consist essentially of, does not consist of, or minimally comprises one or more compounds characterized by a flavor region, a flavor-active region, or a peak, used interchangeably herein, as measured by ultra-high performance liquid chromatography-quadrupole time-of-flight mass spectrometry (UHPLC/QTOF-MS) under the conditions described herein, where the flavor region, the flavor-active region, or the peak elutes at a retention time selected from the group consisting of: ˜5.67 mins, ˜8.202 mins, ˜8.513 mins, ˜8.758 mins, ˜9.002 mins, ˜9.435 mins, and any combinations thereof. See, TABLE 3. In some embodiments, such one or more compounds can be in an amount or concentration below its respective taste or flavor threshold. Retention time (RT) refers to the time taken for the analyte to pass through the column and then detected. Each peak area of Off-flavor and Neutral taste bioBG of TABLE 3 was calculated as follows: relative concentration=area compound/area ISTD. Relative concentration: Off-Flavor vs. Neutral taste bioBG was calculated as follows: [(Area Neutral taste bioBG−Area Off-Flavor)/Area Neutral taste bioBG]*100. Several parameters can affect the retention time, such as but not limited to, temperature, flow, column performance, and the like. Some embodiments are directed to a BG product described here comprising BG (e.g., bioBG), which is characterized by a peak as measured by ultra-high performance liquid chromatography-quadrupole time-of-flight mass spectrometry (UHPLC/QTOF-MS) that elutes at a retention time of ˜1.005 mins.

Additional embodiments provide a BG product comprising a bioBG, where the BG product does not comprise, does not consist essentially of, does not consist of, or minimally comprises one or more compounds characterized by a flavor region, a flavor-active region, or a peak, used interchangeably here, as measured by ultra-high performance liquid chromatography-quadrupole time-of-flight mass spectrometry (UHPLC/QTOF-MS) under the conditions described herein, where the flavor region, the flavor-active region, or the peak elutes at a relative retention time selected from the group consisting of: ˜5.614 mins, ˜8.121 mins, ˜8.429 mins, ˜8.671 mins, ˜8.913 mins, ˜9.341 mins, and any combinations thereof. See, TABLE 3. In some embodiments, such one or more compounds can be in an amount or concentration below its respective taste or flavor threshold.

In some embodiments, a BG product of the disclosure, comprising a bioderived BG (bioBG), where the BG product does not further comprise, does not further consist essentially of, does not further consist of, or minimally comprises one or more compounds having a mass spectrum characterized by ultra-high performance liquid chromatography-tandem mass spectrometry (UHPLC-MS/MS) at a collision energy of 10 V, where the mass spectrum is selected from the group consisting of:
(a) 191.0737 m/z, 172.977 m/z, 141.9585 m/z, 87.0804 m/z, and 69.0700 m/z;
(b) 127.0774 m/z, 109.0650 m/z, and 81.0699 m/z;
(c) 204.1591 m/z, 159.1012 m/z, 141.9584 m/z, 89.0593 m/z, and 60.0803 m/z;
(d) 143.1070 m/z, 103.0756 m/z, 89.0599 m/z, and 71.0493 m/z;
(e) 206.1753 m/z, 143.1070 m/z, 86.0966 m/z, and 60.0807 m/z;
(f) 206.1757 m/z, 161.1178 m/z, 143.1070 m/z, 89.0601 m/z, and 71.0494 m/z;
(g) 103.0751 m/z, 89.0595 m/z, 71.0489 m/z, and 60.0804 m/z;
(h) 310.2595 m/z, 296.2439 m/z, 141.9597 m/z, 100.1129 m/z, 86.0973 m/z, and 60.0815 m/z;
(i) 310.2585 m/z, 296.2432 m/z, 161.1171 m/z, 86.0962 m/z, and 60.0805 m/z; 0) 167.1067 m/z, 141.9587 m/z, 86.0964 m/z, and 60.0806 m/z;
(k) 199.1331 m/z, 141.9586 m/z, 100.1120 m/z, and 86.0964 m/z; and any combinations thereof. Additional embodiments provide a BG product comprising a bioBG mass spectrum of: 91.0756 m/z, 73.0648 m/z, and 55.0541 m/z. See, TABLE 4. In some embodiments, such one or more compounds can be in an amount or concentration below its respective taste or flavor threshold.

Additional embodiments are directed to a BG product of the disclosure, comprising a bioderived BG (bioBG), where the BG product does not further comprise, does not further consist essentially of, does not further consist of, or minimally comprises one or more compounds having a flavor-active region characterized by ultra-high performance liquid chromatography-tandem mass spectrometry (UHPLC-MS/MS) at a collision energy of 10 V, where the flavor-active region comprises a retention time selected from the group consisting of: ˜2.658 mins, ˜4.801 mins, ˜5.493 mins, ˜5.683 mins, ˜6.047 mins, ˜6.237 mins, ˜7.978 mins, ˜8.343 mins, ˜8.817 mins, ˜9.275 mins, ˜13.229 mins, and any combinations thereof, where the retention time of BG is 0.996 min. In some embodiments, such one or more compounds can be in an amount or concentration below its respective taste or flavor threshold.

In some embodiments, the disclosure is directed to a BG product of the disclosure, comprising a bioderived BG (bioBG), where the BG product does not further comprise, does not further consist essentially of, does not further consist of, or minimally comprises one or more compounds having a flavor-active region characterized by ultra-high performance liquid chromatography-tandem mass spectrometry (UHPLC-MS/MS) at a collision energy of 10 V, where the flavor-active region comprises a relative retention time selected from the group consisting of: ˜2.679 mins, ˜4.840 mins, ˜5.537 mins, ˜5.729 mins, ˜6.096 mins, ˜6.287 mins, ˜8.042 mins, ˜8.410 mins, ˜8.888 mins, ˜9.350 mins, ˜13.335 mins, where the relative retention time of BG is 1 min. In some embodiments, such one or more compounds can be in an amount or concentration below its respectivetaste or flavor threshold.

In some embodiments, a BG product of the disclosure comprising a neutral taste bioBG, where the BG product does not comprise, does not consist essentially of, does not consist of, or minimally comprises one or more compounds selected from the group consisting of: 2-methoxymethyl-2,4,5-trimethyl-1,3-dioxolane; toluene; and combinations thereof. In some embodiments, such one or more compounds can be in an amount or concentration below its respective taste or flavor threshold. Further embodiments provide for such one or more compounds that are contributors to “off-flavor”. For example, such one or more compounds, including toluene, can contribute to or be responsible for “off-flavor” in a bioBG product as described here.

Some embodiments provide a BG product of the disclosure, where the BG product comprises 1,3-butylene glycol (BG). In some embodiments, the BG product comprises BG, where the BG is a bioderived BG (bioBG). The BG of the disclosure can be selected from the group consisting of: (R)-1,3-BG; (S)-1,3-BG; a mixture of (R)-1,3-BG and (S)-1,3-BG, and salts, esters, or derivatives thereof. In some embodiments, the BG product of the disclosure comprises a BG that is (R)-1,3-BG or (S)-1,3-BG or a mixture of (R)-1,3-BG and (S)-1,3-BG, tautomers, enantiomers, diastereomers, stereoisomers or mixtures thereof (including racemic mixtures), or pharmaceutically acceptable salts, hydrates, or solvates, thereof, or the like.

Further embodiments provide for any of the described 1,3-BG products or bioBG products comprising or consisting essentially of: (R)-1,3-butylene glycol. Some embodiments provide for a 1,3-BG or a bioBG racemate having more (R)-1,3-BG than (S)-1,3-BG. In another embodiment, the 1,3-BG product, including a bioBG product, comprises (R)-1,3-BG, where (R)-1,3-butylene glycol is present in a 1,3-BG product or a bioBG product in a weight percent of more than or equal to 75% (e.g., 80%, 85%, 90%, 95%; 96%; 97%; 98%; 99%; 99.5%; 99.9%; 100%); where (R)-1,3-butylene glycol is present in a 1,3-BG product or a bioBG product in a weight percent of less than or equal to 100% (e.g., 99.8%, 99.3%, 98.5%, 97.5%, 96.5%, 95.5%, 93%, 87%, 83%, 77%); or (R)-1,3-butylene glycol, where (R)-1,3-butylene glycol is present in a 1,3-BG product or a bioBG product in a weight percent of 75%-100% (e.g., 80%-99%; 85%-95%; 87%-93%) as detected by mechanical separation, reaction with enzymes, formation of diastereomers, or chromatographic methods (e.g., chiral chromatography, gas chromatography, GC/MS, LC-MS, MD-GC, TLC). In another embodiment, a 1,3-BG product or bioBG product comprises (S)-1,3-butylene glycol, where (S)-1,3-butylene glycol is present in a 1,3-BG product or a bioBG product in a weight percent of more than or equal to 0% (e.g., 0.001%, 0.01%, 0.05%, 0.1%, 0.15%, 0.2%, 0.5%, 1%, 2%, 3%, 4%, 5%, 10%, 20%, 25%, 30%); (S)-1,3-butylene glycol, where (S)-1,3-butylene glycol is present in a 1,3-BG product or a bioBG product of in less than or equal to 25% (e.g., 20%, 15%, 12%, 8%, 5.5%, 4.5%, 3.5%, 2.5%, 1.5%, 1.2%, 0.8%, 0.3%, 0.1%, 0.08%, 0.02%, 0.005%, 0.001%); or (S)-1,3-butylene glycol, where (S)-1,3-butylene glycol is present in a 1,3-BG product or bioBG in 0%-25% (e.g., 1%-22%; 2%-12%; 3%-10%; 4%-8%).

In another embodiment, the 1,3-BG product, including a bioBG product, of the disclosure can comprise, consist essentially of, or consist of (S)-1,3-BG. Further embodiments provide a BG or bioBG racemate of the disclosure comprising more (S)-1,3-BG than (R)-1,3-BG in a BG product or a bioBG product described here. In some embodiments, (S)-1,3-butylene glycol is present in a 1,3-BG product or a bioBG product of the disclosure in a weight percent of more than or equal to 75% (e.g., 80%, 85%, 90%, 95%; 96%; 97%; 98%; 99%; 99.5%; 99.9%; 100%); where (S)-1,3-butylene glycol is present in a 1,3-BG product or a bioBG product in a weight percent of less than or equal to 100% (e.g., 99.8%, 99.3%, 98.5%, 97.5%, 96.5%, 95.5%, 93%, 87%, 83%, 77%); or (S)-1,3-butylene glycol, where (S)-1,3-butylene glycol is present in a 1,3-BG product or a bioBG product in a weight percent of 75%-100% (e.g., 80%-99%; 85%-95%; 87%-93%) as detected by mechanical separation, reaction with enzymes, formation of diastereomers, or chromatographic methods (e.g., chiral chromatography, gas chromatography, GC/MS, LC-MS, MD-GC, TLC). In another embodiment, a 1,3-BG product or bioBG product comprises, consists essentially of, or consists of (R)-1,3-butylene glycol, where (R)-1,3-butylene glycol is present in a 1,3-BG product or a bioBG product in a weight percent of more than or equal to 0% (e.g., 0.001%, 0.01%, 0.05%, 0.1%, 0.15%, 0.2%, 0.5%, 1%, 2%, 3%, 4%, 5%, 10%, 20%, 25%, 30%); (R)-1,3-butylene glycol, where (R)-1,3-butylene glycol is present in a 1,3-BG product or a bioBG product of in less than or equal to 25% (e.g., 20%, 15%, 12%, 8%, 5.5%, 4.5%, 3.5%, 2.5%, 1.5%, 1.2%, 0.8%, 0.3%, 0.1%, 0.08%, 0.02%, 0.005%, 0.001%); or (R)-1,3-butylene glycol, where (R)-1,3-butylene glycol is present in a 1,3-BG product or bioBG in 0%-25% (e.g., 1%-22%; 2%-12%; 3%-10%; 4%-8%).

The 1,3-butylene glycol or bioderived butylene glycol (bioBG), provided here can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, the BG or bioBG may exist in various stereoisomeric forms. “Stereoisomers” are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared or obtained as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least, for example, 60%, 65%, 70%, 75%, 80%, 85%, 90%, 95%, 99%, or 99.9% by weight relative to the other stereoisomers. When a single enantiomer is named or depicted by structure, the depicted or named enantiomer is at least, for example, 60%, 70%, 80%, 90%, 99%, or 99.9% by weight optically pure. When a single diastereomer is named or depicted by structure, the depicted or named diastereomer is at least, for example, 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least, for example, 60%, 70%, 80%, 90%, 99%, or 99.9% by mole fraction pure. When a single diastereomer is named or depicted by structure, the depicted or named diastereomer is at least, for example, 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either stereoisomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Some embodiments provide a BG product of the disclosure, where the BG product comprises a BG, and the BG comprises a bioderived BG (bioBG). In some embodiments, the BG product comprises a neutral taste or flavor or minimally comprises undesirable or offensive tastes or flavors. In further embodiments, any BG product described here comprising a neutral taste bioBG can comprise, consist essentially of, consist of, or minimally comprise one or more flavor compounds which can be responsible for a flavor region or a flavor-active region described here, where the one or more compounds can be present in an amount below its respective flavor threshold.

Gustation (or taste) and olfaction (or smell) are highly interconnected senses in that both involve molecules of the stimulus entering the body and bonding to receptors. Accordingly, a person of ordinary skill in the art would appreciate that aromatic compounds can influence taste or flavor. Therefore, in some embodiments, a disclosed BG product comprising a neutral taste or neutral flavor bioBG can comprise, consist essentially of, consist of, or minimally comprise one or more aromatic compounds which can be responsible for a taste-active region or a flavor-active region described here. In some embodiments, such one or more compounds can be present in an amount below its respective taste or flavor threshold. Additional embodiments provide for a neutral taste or flavor bioBG product that comprises, consists essentially of, consists of, or minimally comprises one or more compounds, which can be responsible for or a contributor to a taste/flavor region, a taste- or flavor-active region described here. In further embodiments, such one or more compounds can be present in an amount or concentration below its respective taste or flavor threshold. Some embodiments can be directed to such one or more compounds which can be responsible for or contribute to an undesirable off-taste or off-flavor in a bioBG product or bioBG composition as described here.

### Neutral Taste BG Composition

Some embodiments provide for a BG product of the disclosure comprising a neutral taste that can be included in a composition. For example, a composition of the disclosure comprises a BG product as described here and a physiologically acceptable vehicle (e.g., carrier, diluent, excipient). As used herein, the phrase “physiologically acceptable” means generally safe for ingestion or contact with biologic tissues, particularly at the levels employed. Physiologically acceptable is used interchangeably with physiologically compatible. The compositions of the disclosure comprise physiologically acceptable or compatible components, including but not limited to, neutral taste bioBG products and vehicles (e.g., carrier, diluent, excipient).

In some embodiments, the BG composition of the disclosure comprises a BG product having BG, such as bioBG, and additionally one or more compounds, where the BG product has a neutral taste or minimally offensive or undesirable taste, where the one or more compounds can be present in an amount below its respective taste or flavor threshold. In some embodiments, a BG composition of the disclosure comprises a neutral taste bioBG product having a property selected from the group consisting of: a solvent, a hypoglycemic agent, a fungicide, a bactericide, a preservative, a food additive, a flavor additive, a calorie source, a source of dietary energy, and any combinations thereof. Some embodiments of the disclosure are directed to a BG product comprising bioBG, where the BG product is substantially or essentially neutral in taste or flavor, and does not mask a flavor, add a flavor, or alter a flavor profile of the BG product or of a composition comprising the BG product of the disclosure. Further embodiments provide an essentially neutral taste, neutral flavor, flavorless, or tasteless BG product, including for example, bioBG, having an improved flavor characteristic compared to crude or unpurified bioBG. Furthermore, a bioBG product or a composition comprising the bioBG product of the disclosure has several properties advantageous for consumer use or application. For example, the bioBG product described here is non-irritating, non-sensitizing, non-allergenic, non-greasy, or has any combinations of the aforementioned properties. Compositions of the disclosure comprising a bioBG product described here can have a property selected from the group consisting of: a solvent, a hypoglycemic agent, a fungicide, a bactericide, a preservative, a food additive, a flavor additive, a calorie source, a source of dietary energy, and any combinations of the aforementioned properties, where the bioBG product has a neutral taste or flavor and is non-allergenic.

Some compositions of the disclosure comprising a BG product do not comprise, do not consist essentially of, do not consist of, or minimally comprise one or more compounds characterized by a flavor region, a flavor-active region, or a peak as measured by ultra-high performance liquid chromatography-quadrupole time-of-flight mass spectrometry (UHPLC/QTOF-MS), wherein the peak elutes at an average retention time of: ˜5.67 mins, ˜8.202 mins, ˜8.513 mins, ˜8.758 mins, ˜9.002 mins, ˜9.435 mins, and any combinations thereof. See, TABLE 3. In some embodiments, such one or more compounds can be in an amount or concentration below its respective taste or flavor threshold.

Some embodiments are directed to a BG composition comprising a BG product described here, where the BG product comprises BG (e.g., bioBG) characterized by a peak that elutes at an average retention time of 1.005 mins. The BG composition of the disclosure further minimally comprises one or more compounds characterized by a peak as measured by ultra-high performance liquid chromatography-quadrupole time-of-flight mass spectrometry (UHPLC/QTOF-MS), where the BG product and BG composition have a neutral taste. In some embodiments, a BG composition of the disclosure comprising a BG product, has a neutral taste or a minimally undesirable or offensive taste, and does not comprise, does not consist essentially of, does not consist of, or minimally comprises one or more compounds characterized by a flavor region, a flavor-active region, or a peak, used interchangeably here, as measured by UHPLC/QTOF-MS, where the peak elutes at an average retention time selected from the group consisting of: ˜5.614 mins, ˜8.121 mins, ˜8.429 mins, ˜8.671 mins, ˜8.913 mins, ˜9.341 mins, and any combinations thereof, where the relative retention time of bioBG is 1 min. In some embodiments, such one or more compounds can be in an amount or concentration below its respective taste or flavor threshold.

In some embodiments, a BG composition having a neutral taste or minimally offensive or undesirable taste can comprise a BG product of the disclosure and a vehicle. The BG composition described here comprises a bioBG and do not comprise, consist essentially of, consist of, or minimally comprises one or more compounds having a mass spectrum characterized by ultra-high performance liquid chromatography-tandem mass spectrometry (UHPLC-MS/MS) at a collision energy of 10 V, where the mass spectrum is selected from the group consisting of:
(a) 191.0737 m/z, 172.977 m/z, 141.9585 m/z, 87.0804 m/z, and 69.0700 m/z;
(b) 127.0774 m/z, 109.0650 m/z, and 81.0699 m/z;
(c) 204.1591 m/z, 159.1012 m/z, 141.9584 m/z, 89.0593 m/z, and 60.0803 m/z;
(d) 143.1070 m/z, 103.0756 m/z, 89.0599 m/z, and 71.0493 m/z;
(e) 206.1753 m/z, 143.1070 m/z, 86.0966 m/z, and 60.0807 m/z;
(f) 206.1757 m/z, 161.1178 m/z, 143.1070 m/z, 89.0601 m/z, and 71.0494 m/z;
(g) 103.0751 m/z, 89.0595 m/z, 71.0489 m/z, and 60.0804 m/z;
(h) 310.2595 m/z, 296.2439 m/z, 141.9597 m/z, 100.1129 m/z, 86.0973 m/z, and 60.0815 m/z;
(i) 310.2585 m/z, 296.2432 m/z, 161.1171 m/z, 86.0962 m/z, and 60.0805 m/z; 0) 167.1067 m/z, 141.9587 m/z, 86.0964 m/z, and 60.0806 m/z;
(k) 199.1331 m/z, 141.9586 m/z, 100.1120 m/z, and 86.0964 m/z; and any combinations thereof. Additional embodiments provide a BG product comprising a bioBG mass spectrum of: 91.0756 m/z, 73.0648 m/z, and 55.0541 m/z. See, TABLE 4. In some embodiments, such one or more compounds can be in an amount or concentration below its respective taste or flavor threshold.

Some embodiments comprise a composition or formulation described here comprising a BG product of the disclosure, where the BG product comprises a bioderived BG (bioBG), and the BG product does not further comprise, does not further consist essentially of, does not further consist of, or minimally comprises one or more compounds having a flavor-active region characterized by ultra-high performance liquid chromatography-tandem mass spectrometry (UHPLC-MS/MS) at a collision energy of 10 V, where the flavor-active region comprises a retention time selected from the group consisting of: ˜2.658 mins, ˜4.801 mins, ˜5.493 mins, ˜5.683 mins, ˜6.047 mins, ˜6.237 mins, ˜7.978 mins, ˜8.343 mins, ˜8.817 mins, ˜9.275 mins, ˜13.229 mins, and any combinations thereof. In some embodiments, such one or more compounds can be in an amount or concentration below its respective taste or flavor threshold.

In some embodiments, the disclosure is directed to a composition or formulation comprising a BG product of the disclosure comprising a bioderived BG (bioBG), where the BG product does not further comprise, does not further consist essentially of, does not further consist of, or minimally comprises one or more compounds having a flavor-active region characterized by ultra-high performance liquid chromatography-tandem mass spectrometry (UHPLC-MS/MS) at a collision energy of 10 V, where the flavor-active region comprises a relative retention time selected from the group consisting of: ˜2.679 mins, ˜4.840 mins, ˜5.537 mins, ˜5.729 mins, ˜6.096 mins, ˜6.287 mins, ˜8.042 mins, ˜8.410 mins, ˜8.888 mins, ˜9.350 mins, ˜13.335 mins, where the relative retention time of BG is 1 min. In some embodiments, such one or more compounds can be in an amount or concentration below its respective taste or flavor threshold.

In some embodiments, provided here is a composition or formulation containing a BG product of the disclosure comprising a neutral taste bioBG, where the BG product does not comprise, does not consist essentially of, does not consist of, or minimally comprises one or more compounds selected from the group consisting of: 2-methoxymethyl-2,4,5-trimethyl-1,3-dioxolane; toluene; and combinations thereof, and comprises a vehicle (e.g., a carrier, a diluent, an excipient, or any combinations thereof. In some embodiments, such one or more compounds can be in an amount or concentration below its respective taste or flavor threshold. Further embodiments provide for such one or more compounds that are contributors to “off-flavor”. For example, such one or more compounds, including toluene, can contribute to or be responsible for “off-flavor” in a bioBG product as described here.

In some embodiments, the composition or formulation described here comprises a BG product of the disclosure, where the composition is a food product. A “food product” as used here means a substance, also including beverages, for consumption by a subject. As used herein, the term “subject” or “consumer” refers to any organism to which a composition in accordance with the disclosure may be applied or administered, e.g., for experimental, diagnostic, organoleptic, or cosmetic purposes. Typical subjects include any animal (e.g., mammals such as humans, dogs, cats, monkeys, mice, rats, rabbits, and non-human primates, etc.).

In some embodiments of the disclosure, a composition or formulation can comprise any of the 1,3-butylene glycol (BG) products described here and a physiologically acceptable vehicle (e.g., carrier, diluent, excipient). Additional embodiments provide for such compositions or formulations, where the BG product comprises a neutral taste or a neutral flavor. In other embodiments, the compositions or formulations comprise a BG product of the disclosure, where the BG product comprises a property selected from the group consisting of or can be used as: a solvent, a hypoglycemic agent, a fungicide, a bactericide, a preservative, a food additive, a calorie source, and any combinations thereof.

Additional embodiments provide a composition or a formulation comprising any of the BG products disclosed here, where the BG product comprising a bioderived BG (bioBG) does not comprise further compounds characterized by flavor-active regions or peaks as measured by ultra-high performance liquid chromatography-quadrupole time-of-flight mass spectrometry (UHPLC/QTOF-MS), wherein the peaks elute at an average retention time of: ˜5.67 mins, ˜8.202 mins, ˜8.513 mins, ˜8.758 mins, ˜9.002 mins, and ˜9.435 mins. In some embodiments, such one or more compounds can be in an amount or concentration below its respective taste or flavor threshold.

Compositions or formulations comprising the bioBG products of the disclosure can be in the form of a food product. Food products include any substance for consumption by a subject or a consumer (e.g., mammals, humans), including food or beverages containing bioBG used as a solvent, a hypoglycemic agent, a fungicide, a bactericide, a preservative, a food additive, a calorie source, and any combinations thereof. Non-limiting examples of food products include flavored beverages, flavored foods, high energy beverages, high energy foods, pharmaceuticals, nutraceuticals, and supplements.

Some embodiments are directed to BG compositions or formulations of the disclosure comprising any of the described BG products, such as bioderived BG (bioBG). In additional embodiments, the BG compositions or formulations of the disclosure can provide a source of food energy and calories for those who are deficient. The BG compositions or formulations of the disclosure can also provide nutritional supplements and/or nutritional replacements.

In some embodiments, the disclosure provides an effective amount of a neutral taste 1,3-BG (e.g., bioBG) product and one or more ingredients (including but not limited to a physiologically acceptable vehicle) of a bioBG composition of the disclosure. The term “effective amount” of a neutral taste bioBG in a composition, such as a high energy food product, for use in increasing energy or calories in a subject who consumes the composition of the disclosure, is an amount sufficient to effect desired results, such as an increase in energy, an increase in calories, a decrease in weight gain, and the like, and, as such, an “effective amount” depends upon the context in which it is being applied. The percent of an effective amount of a neutral taste bioBG product in total composition can be in the form of weight/volume % (wt/vol % or w/v %), weight/weight % (wt/wt % or w/w %), or volume/volume % (vol/vol % or v/v %), where the amount (weight or volume) of a neutral taste bioBG product is expressed as a percentage of the total composition weight or volume. The amount of neutral taste bioBG in a composition of the disclosure (e.g., food product) can include percentages of greater than or equal to 0.01% by weight (e.g., 0.06%; 0.11%; 0.16%; 0.21%; 0.26%; 0.31%; 0.36%; 0.41%; 0.46%; 0.51%; 0.56%; 0.61%; 0.66%; 0.71%; 0.76%; 0.81%; 0.86%; 0.91%; 0.96%; 1%; 1.05%; 1.25%; 1.5%; 1.75%; 2%; 2.5%; 3%; 3.5%; 4%; 4.5%; 5%; 5.5%; 6%; 6.5%; 7%; 7.5%; 8%; 8.5%; 9%; 9.5%; 10%; 10.5%; 15%; 20%; 25%; 30%; 35%; 40%; 45%; 50%; 55%; 60%); less than or equal to 50% (e.g., 45%; 40%; 35%; 30%; 25%; 20%; 15%; 10%; 9.5%; 8%; 7%; 6%; 5%; 4%; 3%; 2%; 1.75%; 1.25%; 1%; 0.75%; 0.25%; 0.17%; 0.12%; 0.07%; 0.02%; 0.01%); or percentages in a range of 0.01%-50% (e.g., 0.05%-45%; 0.1%-30%; 1%-20%; 3%-15%; 5%-10%).

In further embodiments, compositions or formulations described here comprising a neutral taste bioBG of the disclosure, can be in the form of a solid or in the form of a liquid composition or a gel. Suitable solid forms of the composition include a bar or powder suitable for mixing with a liquid, for example water, milk, fruit juice, or the like, at the point of use. Suitable forms of liquid composition include for example a syrup, an emulsion, and a suspension. When in the form of a syrup, the composition can further contain as a physiologically acceptable vehicle, for example, saccharose or saccharose with glycerol and/or mannitol and/or sorbitol. When in the form of a suspension or emulsion, the composition can contain as a physiologically acceptable vehicle, for example, a natural gum, agar, sodium alginate, pectin, methylcellulose, carboxymethylcellulose or polyvinyl alcohol.

A food product, such as an edible or drinkable material, can be composed of one or more of the macronutrients: proteins, carbohydrates, fats, or combinations thereof, which is used in the body of a subject to sustain growth, repair damage, aid vital processes and/or furnish energy. A food product can also contain one or more micronutrients, such as vitamins or minerals, or additional dietary ingredients such as flavoring additives and dyes or color additives. The term food product as used herein also covers a beverage.

In some embodiments, the compositions or formulations of the disclosure can be a nutraceutical. A nutraceutical can be a food ingredient, food supplement, or food product which is considered to provide a medical or health benefit, including the prevention and treatment of disease. A nutraceutical can be adapted to confer a particular health benefit on the consumer. A nutraceutical can comprise a micronutrient, such as a vitamin, mineral, herb, or phytochemical at a higher level than would be found in a corresponding regular food product. The level can be selected to optimize an intended health benefit of the nutraceutical when consumed either as a single serving or as part of a diet regimen or course of nutritional therapy.

Further embodiments provide bioBG compositions, where the composition comprises a physiologically acceptable vehicle (e.g., carriers, diluents, excipients) containing additional components or ingredients that can be included in a composition of the disclosure, where the physiologically acceptable vehicle can be selected from the group consisting of: carriers: saccharose, saccharose with glycerol and/or mannitol and/or sorbitol, natural gum, agar, sodium alginate, pectin, methylcellulose, carboxymethylcellulose, polyvinyl alcohol; diluents: lactose, dextrose, saccharose, cellulose, starch (e.g., corn starch, potato starch); lubricants: silica, talc, stearic acid, magnesium or calcium stearate and/or polyethylene glycols; binding agents: starches, arabic gums, gelatin, methylcellulose, carboxymethylcellulose, polyvinyl pyrrolidone; disintegrating agents: starch, alginic acid, alginates or sodium starch glycolate; effervescing agent; dyes or colorants; sweeteners; wetting agents: lecithin, polysorbates, lauryl sulphates; and any combinations thereof.

In additional embodiments, a bioBG composition described here is an energy drink comprising a neutral taste or neutral flavor bioBG product comprising a bioBG of the disclosure. Some embodiments provide a bioBG energy drink composition comprising (R)-1,3-BG, tautomers, enantiomers, diastereomers, stereoisomers or mixtures thereof (including racemic mixtures), or pharmaceutically acceptable salts, hydrates, or solvates, thereof, or the like. The energy drink described here can further comprise flavoring substances, flavoring additives, or dyes. The amount of neutral taste bioBG present in a BG energy drink composition of the disclosure can include, but is not limited to, 1 mg neutral taste bioBG per ounce of the energy drink composition or greater (e.g., 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200); 300 mg/oz or less (e.g., 290, 280, 270, 260, 250, 240, 230, 220, 210, 205, 195, 185, 175, 165, 155, 145, 135, 125, 115, 95, 85, 75, 65, 55, 45, 35, 25, 19.5, 18.5, 17.5, 16.5, 15.5, 14.5, 13.5, 12.5, 11.5, 10.5, 9.5, 8.5, 7.5, 6.5, 5.5, 4.5, 3.5, 2.5, 1.5); 1 mg/oz to 300 mg/oz (e.g., 1-300; 10-295; 20-285; 30-275; 40-265; 50-255; 60-245; 70-235; 80-225; 90-215; 100-205; 110-195; 120-185; 130-175; 140-165; 150-155), where the neutral taste bioBG does not comprise, does not consist essentially of, or does not consist of a biting, irritating bitterness.

### Uses and Methods of Using Neutral Taste BG Compositions

In some embodiments of the disclosure, a method can comprise increasing energy (e.g., calories) in a subject or consumer in need thereof, where increasing energy occurs by the subject consuming a composition containing a BG product (e.g., neutral taste bioBG product) of the disclosure. A subject or consumer in need thereof is typically a subject who consumes a food product, including beverages, food supplements, dietary supplements, or nutraceuticals, where the food product is an edible or drinkable substance that furnishes energy, is used in vital processes for sustaining life and growth, and repairs damage. For example, a food product can be any substance for animal (e.g., human) consumption, such as flavored beverages, flavored foods, high energy beverages, high energy foods, pharmaceuticals, nutraceuticals, supplements, and the like.

Methods as described here can comprise a subject who consumes a food product composition containing a BG product (e.g., neutral taste bioBG product), of the disclosure where non-limiting examples of such compositions include flavored beverages, flavored foods, high energy beverages, high energy foods, pharmaceuticals, nutraceuticals, and supplements. The methods of the disclosure can include a subject consuming a composition containing a neutral tasting BG product described here to result in an increase in energy in the subject. Additional methods of the disclosure can comprise administering a BG composition to a subject, where the method is for increasing energy or decreasing weight gain in the subject, where the BG composition comprises a neutral taste bioBG food product. In some embodiments, the methods described here can provide an increase of energy, for example, 5 kcal/g or more (e.g., 6 kcal/g, 7 kcal/g, 8 kcal/g, 9 kcal/g, 10 kcal/g) in a subject. Other embodiments of the disclosure provide methods that decrease weight gain in a subject comprising consuming a composition containing a neutral tasting BG product described here.

Some embodiments provide a use of the 1,3-butylene glycol (BG) product described here or the composition comprising any one of the BG products described here for increasing energy, such as calories, in a subject in need thereof.

In additional embodiments, use of the 1,3-butylene glycol (BG) product described here or any of the compositions described here for increasing energy or decreasing weight gain in a subject in need thereof is provided. The use can be in a food product, where the BG product (e.g., neutral taste bioBG product) or the composition comprising the BG product (e.g., neutral taste bioBG product) is added to the food product. Further embodiments are directed to the use of a neutral taste bioBG product or composition comprising the neutral taste bioBG product, where the neutral taste bioBG product or composition comprising the neutral taste bioBG product are in a food product, such as but not limited to: flavored beverages, flavored foods, high energy beverages, high energy foods, pharmaceuticals, nutraceuticals, supplements, or any combinations thereof.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage inclusive of the lowermost and uppermost range, and integrals therebetween, such as for example, 0.1%, 1%, 1.5%, 2.0%, 2.5%, and 3%. Additionally, a range of 0.1 to 3% includes, for example, subsets of the original range including from 0.5% to 2.5%, from 1% to 3%, from 0.1% to 2.5%, etc. It will be understood that the sum of all weight percents (weight %) of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented.

### EXAMPLES

The following examples illustrate specific aspects of the instant description. The examples should not be construed as limiting, as the example merely provides specific understanding and practice of the embodiments and its various aspects.

### Example 1: Process of Obtaining Neutral Taste Bioderived 1,3-Butylene Glycol

### Method to Produce Neutral Taste Sample DSP-555-2

Bioderived 1,3-butylene glycol (bioBG) was produced and deodorized using a caustic treatment followed by distillation described here to produce a neutral taste bioBG product 1 of the disclosure (DSP-555-2). The block flow diagram which describes the process implemented to produce 800 kgs of deodorized bioBG. See, FIG. 1.

Odorous bioBG was treated with a caustic solution (˜40 wt %-˜50 wt % NaOH), such that the final concentration of NaOH in the reaction solution was 0.25 wt %. This mixture was then reacted in a tank vessel (base reactor) at 120° C. for 20 mins while stirring and as a result, impurities, such as ketones and aldehydes, convert to other less volatile materials. Alternatively, the continuous flow reactor with heat exchanger could be used to carry out the reaction.

The reaction product was then fed into a WFE (wiped film evaporator) for degassing and water removal. This helps in reaching low vacuum in subsequent steps. The bottom stream from the WFE was then fed into a SPE (short pass evaporator) for base removal along with some heavies. This step was typically run at a relatively milder temperature (˜90° C.) and a short residence time (in seconds) to avoid formation of degradation products.

The lights vapor stream from the SPE was then condensed and fed into the 1st distillation column, equipped with a wiped film evaporator (WFE) as a reboiler, where residual heavies are removed as bottoms under vacuum (10-20 torr) at 100° C.-110° C. The tops from this step was sent to a 2^{nd} distillation column of the same size where the lights components were distilled off and a low odor BG and neutral taste BG (DSP-555-2) was drawn from the bottom. The pressure in this column was low, 10 torr-20 torr at 120° C.; however, had a higher reflux ratio than the heavies removal step for better separation.

### Method to Produce Neutral Taste Sample DSP-555-4

Crude high odor bioderived 1,3-butylene glycol (bioBG) was produced and deodorized using the caustic distillation process described here to produce a bioBG Product 2 (DSP-555-4).

### Crude Analysis

The crude, BIO-BG Natural Butylene Glycol (D2413) was received in 4×500 mL glass bottles. The odor of the crude was noticeable. This high odor bioBG was deodorized as described here:

| TEST | RESULT |
|---|---|
| Water | 0.04% |
| 1,3-butanediol | 99.6781%* |
| GC Lights | 0.1456%* |
| GC Heavies | 0.1336%* |
| Acidity (as acetic acid) | 0.0027% |

*GC results normalized for water and acidity

### Distillation Details

1,3-Butanediol (1,731.8 g) (bioBG) was charged to a 2 L 3 necked flask and placed under an 24 theoretical plate column fitted with a poly(tetrafluorethylene) (PTFE) (0.25 mm ID) nitrogen sparge. The temperature was monitored using a PTFE thermocouple in the base and a stainless steel thermocouple in the side stream and overheads which were connected to a RS52 2channel K handheld thermometer. After 18 theoretical plates, a side stream was fitted and was connected to a Perkin triangle and 250 ml round bottom flask. A reflux controller and Perkin triangle fitted with a 250 ml round bottom flask were used as overheads. A vacuum of 20 mmHg was applied to the system using a Javac DS75 high vacuum pump. The system was brought to a boil and held at a total reflux for ˜15 minutes. After this, because the temperatures at the side stream and at the column top were similar, the top reflux ratio was set to 1:1 to remove any light boilers for ˜45 minutes. The side stream was then set to take off 1,3-butanediol at 1:1 and the reflux ratio adjusted to ˜10:1. The purity and the odor of the side stream was unacceptable, so the side stream take off was stopped while more lights were removed from the column top at a reflux ratio of ˜15:1 or ˜10:1. On start up the following day, the side stream was set to take off 1,3-butanediol at 1:1 and the top reflux ratio set to take off at 5:1. The reflux ratio at the column top was varied slightly through the distillation to determine if the odor and purity of the side stream improved with an increased reflux ratio. The rest of the distillation was run with a top reflux ratio of ˜5:1 and a side stream take off of 1:1. Towards the end of the distillation, the GC heavies in the side stream began to increase and knocked the purity of the side stream below the specification of a 99.5% minimum.

### Distillation Run Sheet

The middle temperature (MT) read low during the run sheet until Apr. 7, 2021 (07/04/2021), which was due to a lack of lagging on the side stream portion of the column.

| | | | | | | | | |
|---|---|---|---|---|---|---|---|---|
| | BT | MT | TT | Middle | Top | Pressure | | Observations/ |
| Time | (° C.) | (° C.) | (° C.) | RR | RR | (mmHg) | Fraction | Comments |

30 Mar. 2021

| | | | | | | | | |
|---|---|---|---|---|---|---|---|---|
| 08:30 | 16.9 | 17.9 | 18.3 | Total | Total | 21 | | Heat on. |
| 09:33 | 93.1 | 18.3 | 18.4 | Total | Total | 20 | | |
| 10:24 | 121.5 | 85.4 | 19.4 | Total | Total | 20 | | |
| 10:54 | 122.8 | 108.7 | 86.0 | Total | Total | 20 | | |
| 11:03 | 123.0 | 111.3 | 118.9 | Total | Total | 20 | | |
| 11:14 | 122.6 | 110.9 | 119.7 | Total | Total | 20 | | TR → 2:1 |
| 12:00 | 121.8 | 114.2 | 119.3 | Total | 1:1 | 20 | T1 (22.9 g) | |
| 12:41 | 122.1 | 113.9 | 119.2 | Total | 2:1 | 20 | T2 (24.3 g) | SS flush |
| 12:49 | 122.4 | 113.9 | 119.0 | Total | 2:1 | 20 | SS1 (9.8 g) | MR → 2:1 |
| 13:34 | 121.7 | 113.2 | 118.5 | 2:1 | 2:1 | 20 | SS2 (16.5 g) | TR → 10:1 |
| | | | | | | | T3 (28.0 g) | |
| 14:23 | 121.2 | 112.4 | 117.1 | 2:1 | 13:1 | 19 | SS3 (13.0 g) | MR → Total |
| | | | | | | | | Take more tops |
| 14:35 | 120.7 | 112.8 | 117.4 | Total | 10:1 | 19 | T4 (13.1 g) | |
| 15:17 | 120.4 | 112.1 | 116.8 | Total | 6:1 | 19 | | TR → 3:1 |
| 15:56 | 120.4 | 113.1 | 117.2 | Total | 3:1 | 19 | T5 (26.7 g) | Heat off. |

31 Mar. 2021

| | | | | | | | | |
|---|---|---|---|---|---|---|---|---|
| 07:54 | 25.3 | 36.3 | 30.6 | Total | Total | | | Heat & vacuum on. |
| 08:49 | 121.6 | 113.6 | 20.6 | Total | Total | 20 | | |
| 09:02 | 122.2 | 116.7 | 119.4 | Total | Total | 20 | | Refluxing. |
| 09:26 | 122.4 | 117.4 | 119.6 | Total | Total | 20 | | TR → 5:1 |
| 10:26 | 123.6 | 119.2 | 120.9 | Total | 2:1 | 20 | SS4 (5.1 g) | SS flush (SS4) |
| | | | | | | | | MR → 2:1 |
| 11:15 | 123.1 | 117.8 | 119.7 | 1:1 | 5:1 | 20 | SS5 (16.9 g) | |
| 12:03 | 128.3 | 123.6 | 119.6 | 1:1 | 5:1 | 20 | SS6 (13.4 g) | |
| | | | | | | | T6 (58.3 g) | |
| 13:02 | 126.2 | 120.9 | 119.6 | 1:1 | 3:1 | 20 | SS7 (25.2 g) | |
| 13:32 | 124.3 | 119.2 | 119.5 | 1:1 | 2:1 | 20 | T7 (40.1 g) | |
| 13:47 | 123.9 | 118.8 | 119.7 | 1:1 | 3:1 | 20 | SS8 (12.4 g) | MR → Total |
| | | | | | | | | TR → 2:1 |
| 14:10 | 127.4 | 121.8 | 120.0 | Total | 2:1 | 20 | T8 (22.2 g) | |
| 14:46 | 126.2 | 121.3 | 120.1 | Total | 2:1 | 20 | T9 (23.2 g) | |
| 15:36 | 127.6 | 122.3 | 120.0 | Total | 2:1 | 20 | T10 (33.8 g) | |
| 16:02 | 126.2 | 121.1 | 120.2 | Total | 2:1 | 20 | | MR → 1:1 |
| 16:35 | 127.7 | 121.9 | 120.0 | 1:1 | 2:1 | 20 | SS9 (24.9 g) | Odour better, |
| | | | | | | | | purity 99.6% |
| | | | | | | | | TR → 10:1 |
| 17:08 | 127.8 | 122.2 | 119.4 | 1:1 | 10:1 | 22 | SS10 (34.4 g) | |
| 17:31 | 124.2 | 118.2 | 119.6 | 1:1 | 10:1 | 22 | SS11 (30.5 g) | Heat off. |
| | | | | | | | T11 (50.8 g) | |

1 Apr. 2021

BS1 (46.2 g)
Taken for GC, odor
analysis and potential
chemical treatment
for odor removal.

6 Apr. 2021

| | | | | | | | | |
|---|---|---|---|---|---|---|---|---|
| 09:07 | 13.9 | 16.1 | 16.4 | Total | Total | | | Heat & vacuum on. |
| 10:09 | 124.1 | 115.9 | 18.9 | Total | Total | 20 | | |
| 10:19 | 124.4 | 117.2 | 121.7 | Total | Total | 20 | | Refluxing. |
| 10:37 | 127.1 | 119.9 | 121.6 | Total | Total | 20 | | TR → 10:1 |
| 11:06 | 131.6 | 123.6 | 121.7 | Total | 10:1 | 20 | | MR →1:1 |
| 11:46 | 134.1 | 127.2 | 121.7 | 1:1 | 8:1 | 20 | SS12 (27.4 g) | |
| 12:17 | 125.6 | 118.7 | 121.5 | 1:1 | 5:1 | 20 | SS13 (36.7 g) | |
| 13:39 | 125.0 | 117.8 | 121.4 | 1:1 | 5:1 | 20 | SS14 (87.2 g) | |
| 14:39 | 125.5 | 118.5 | 121.5 | 1:1 | 5:1 | 20 | SS15 (106.6 g) | |
| | | | | | | | T12 (50.0 g) | |
| 16:02 | 124.7 | 117.6 | 121.2 | 1:1 | 5:1 | 20 | SS16 (149.1 g) | Heat off. |

7 Apr. 2021

| | | | | | | | | |
|---|---|---|---|---|---|---|---|---|
| 09:28 | 14.4 | 17.2 | 17.7 | Total | Total | 20 | | Heat on. |
| 10:46 | 122.5 | 115.2 | 119.7 | Total | Total | 20 | | Refluxing. |
| 10:59 | 122.8 | 115.9 | 120.3 | Total | Total | 20 | | TR → 10:1 |
| | | | | | | | | MR → 1:1 |
| 11:25 | 122.8 | 115.9 | 120.3 | 1:1 | 10:1 | 20 | SS17 (34.3 g) | Lagging added |
| | | | | | | | | to SS. |
| 12:18 | 127.2 | 123.1 | 119.7 | 1:1 | 10:1 | 20 | SS18 (100.4 g) | |
| 13:03 | 127.2 | 122.3 | 119.7 | 1:1 | 5:1 | 20 | SS19 (114.2 g) | MR → Total |
| | | | | | | | | SS19 purity |
| | | | | | | | | 99.2% |
| 13:46 | 126.8 | 122.3 | 119.7 | Total | 5:1 | 20 | T13 (42.4 g) | MR → 1:1 |
| 15:17 | 124.9 | 119.6 | 119.6 | 1:1 | 5:1 | 20 | SS20 (204.1 g) | Heat off. |

BT—bottom temperature;
MT—middle temperature;
TT—top temperature;
RR—reflux ratio;
SS—side stream;
T—tops

### Fraction Analysis as GC Area (%)

1,3-Butanediol is quite viscous and significant variation was seen in the repeat injection of the crude sample. For this reason, all GC analysis was performed as a 50:50 mixture with water.

Analysis of the crude and composite was performed neat. The syringe was thoroughly washed with water between each run.

| | Mass | % GC | % 1,3-Bu- | % GC | |
|---|---|---|---|---|---|
| Fraction | (g) | Lights | tanediol | Heavies | Odour |
| Crude * | 1731.8 | 0.0521 | 98.5535 | 1.3517 | |
| SS1 | 9.8 | 0.1769 | 99.4564 | 0.3667 | smelly |
| SS2 | 16.5 | 0.0129 | 99.1592 | 0.8279 | smelly |
| SS3 | 13.0 | 0.0106 | 99.3232 | 0.6662 | smelly |
| SS4 | 5.1 | 0.0163 | 99.6661 | 0.3176 | smelly |
| SS5 | 16.9 | 0.0061 | 99.6748 | 0.3191 | smelly |
| SS6 | 13.4 | 0.0046 | 99.1464 | 0.8490 | smelly |
| SS7 | 25.2 | 0.0131 | 98.3314 | 1.6555 | smelly |
| SS8 | 12.4 | 0.0088 | 99.1968 | 0.7944 | milder |
| SS9 | 24.9 | 0.0065 | 99.6453 | 0.3482 | milder |
| SS10 | 34.4 | 0.0048 | 99.5572 | 0.4380 | mild |
| SS11 | 30.5 | 0.0044 | 99.6370 | 0.3586 | mild |
| SS12 | 27.4 | 0.0097 | 99.4827 | 0.5076 | mild |
| SS13 | 36.7 | 0.0026 | 99.5393 | 0.4581 | very low |
| SS14 | 87.2 | 0.0021 | 99.7069 | 0.2910 | mild |
| SS15 | 106.6 | 0.0028 | 99.5918 | 0.4054 | mild |
| SS16 | 149.1 | 0.0013 | 99.6303 | 0.3684 | mild |
| SS17 | 34.3 | 0.0043 | 99.7037 | 0.2920 | mild |
| SS18 | 100.4 | 0.0025 | 99.2659 | 0.7316 | slightly |
| | | | | | fruity? |
| SS19 | 114.2 | 0.0027 | 99.3554 | 0.6419 | mild |
| SS20 | 204.1 | 0.0011 | 99.4540 | 0.5449 | mild |
| T1 | 22.9 | 0.9946 | 96.3821 | 2.6233 | |
| T2 | 24.3 | 0.3910 | 97.6079 | 2.0011 | |
| T3 | 28.0 | 0.1349 | 97.0450 | 2.8201 | |
| T4 | 13.1 | 0.1222 | 98.9421 | 0.9357 | |
| T5 | 26.7 | 0.0348 | 98.8430 | 1.1222 | |
| T6 | 58.3 | 0.0167 | 99.2596 | 0.7237 | |
| T7 | 40.1 | 0.0050 | 98.9969 | 0.9981 | |
| T8 | 22.2 | 0.0035 | 99.4025 | 0.5940 | |
| T9 | 23.2 | 0.0044 | 99.2474 | 0.7482 | |
| T10 | 33.8 | 0.0045 | 99.2555 | 0.7400 | |
| T11 | 50.8 | 0.0004 | 99.7298 | 0.2698 | |
| T12 | 50.0 | 0.0080 | 99.6148 | 0.3772 | |
| T13 | 42.4 | 0.0059 | 99.7772 | 0.2169 | |
| T14 | 20.8 | 0.0007 | 99.8123 | 0.1870 | |
| BS1 | 46.2 | | | | |
| Residues | 112.2 | n/d | 99.7989 | 0.2011 | |

* GC results normalized for water and acidity.

### Residues

The residues (112.2 g) consisted of a pale yellow colored liquid containing no solids. The residues had no significant odor.

The walls of the flask were clean once the residues were decanted, a cold water wash was used to clean the flask successfully.

### Composite Sample

A composite sample (SS 9-20, 949.8 g) was prepared and analyzed as follows:

| Test | Result | Specification | |
|---|---|---|---|
| Water | 0.02% | | |
| 1,3-Butanediol | 99.9238% * | 99.5% | min. |
| GC Lights | 0.0150% * | | |
| GC Heavies | 0.0392% * | | |
| Color | 0 Hazen | 10 Hazen max. | |
| Appearance | Clear Colorless Liquid | Clear Colorless Liquid | |
| SG (at 20° C.) | 1.005 | 1.004 to 1.006 | |
| Odor | Odorless/Low Odor | Odorless | |
| Free acid (as acetic acid) | 0.002% | 0.005% | max. |

* GC results normalized for water and acidity.

### Mass Balance

| | Weight (g) | Percentage (%) |
|---|---|---|
| Total Mass In | 1731.8 | 100.00 |
| Spec 13BD (SS) | 949.8 | 54.84 |
| Non spec 13BD (SS) | 112.3 | 6.48 |
| Lights/Tops | 456.6 | 26.37 |
| Residues | 112.2 | 6.48 |
| Base Samples | 46.2 | 2.67 |
| Losses | 54.7 | 3.16 |

### Conclusion

The odor of the lights fractions all had a significant odour. The odor of the early side stream fractions also had a significant odor which eventually reduced to an acceptable level. A lot of the charge was taken as tops and off-spec side stream, this resulted in a poor yield of ˜55%. Throughout the distillation, the reflux ratio at the column top had to be altered to keep the side stream purity on spec.

The yield of the distillation can be improved upon if repeated by increasing the top reflux ratio to 15:1, on the plant there is also the opportunity to recycle inters type material which would increase the yield. To improve the yield in the lab, the side stream take off should not start until the purity in the tops fractions was >99%, which can occur by slowly removing light boilers at a higher reflux ratio of ˜15:1.

### Corrosion Studies

A 321 corrosion coupon was placed in the flask during the distillation of 1,3-butanediol. The corrosion rate was deemed acceptable. The coupon showed no weight loss.

### Other Work

BS1 was taken, split, and treated with Venpure (0.7%), NaOH (10%) and NaBH₄ (0.5%) to determine whether any improvement in odor was possible. The samples were mixed and placed in the oven for a short period of time before their odor was checked using the hot water test (20 mL hot water+1 mL sample). The general opinion was that there was no odor improvement observed.

It was difficult to conclude whether any of these treatment options would provide a better odor without running through the entire distillation process.

### Analytical Details

The following equipment and conditions were used to analyze the distillation fractions:

| | |
|---|---|
| Column Type/Dimensions | HP-1; 30 m × 0.32 mm |
| | (film thickness 1.0 μl) |
| Carrier Gas/Pressure | Hydrogen/5.0 psig |
| Injector/Detector Temps | 250° C./270° C. |
| Sample size | 0.1 μl |
| Temp. Programme | 80° C. to 260° C. at |
| | 20° C./min (iso 11 mins) |
| Total Run Time | 20 Minutes |
| Split Ratio | 20:1 |
| GC | GC-3 |
| Method File | 1,2-Pentanediol |
| Method Name | CC5_HP1 |

The following equipment and conditions were used to analyze the spec composite and crude samples:

| | |
|---|---|
| Column Type/Dimensions | CP7834: CP-Sil 24CB; 30 m × |
| | 0.32 mm (film thickness 0.5 μl) |
| Carrier Gas/Pressure | Hydrogen/5.0 psig |
| Injector/Detector Temps | 250° C./270° C. |
| Sample size | 1.0 μl |
| Sample preparation | 10 drops sample: 1 mL HPLC methanol, |
| | mixed well before injection |
| Temp. Programme | 80° C. to 240° C. at 20° |
| | C./min (iso 11 mins) |
| Total Run Time | 20 Minutes |
| Split Ratio | Splitless |
| Integration Notes | Lock out methanol peak |
| GC | GC-3 |
| Method File | 1,3-Butylene Glycol |
| Method Name | CIQC 210328 |

### Equipment

This distillation work used the ‘new’ side stream column which was installed in 2020. The ‘new’ and ‘old’ side stream columns were set up very similarly. Both side stream columns had an 18-plate column fitted with a side stream and a 6-plate column on top of that.

The differences between the columns are that the ‘old’ side stream used knit mesh packing and the ‘new’ side stream used Sulzer packing. The ‘old’ side stream also had column heating fitted to the lower 18-plate and upper 6-plate column. The ‘new’ side stream only had column heating fitted to the lower 18-plate column. For this reason, the boil up achieved in the upper column of the ‘new’ side stream was not as good as the ‘old’ side stream, meaning a slower reflux and distillation.

The ‘new’ side stream column was then fitted with temporary column heating on the upper column to help minimize differences between the columns.

### Specifications

The specifications of a number of suppliers were compared and an initial specification was chosen for the work described here.

The table below details the specifications for 1,3-butanediol for various suppliers and the target specification aimed to meet. The market leader is Diacel. The selected target specification was the same as Godavari's but with a tighter acidity spec of 0.005% max. as acetic acid.

| | | | | | | | Nihon | | | |
|---|---|---|---|---|---|---|---|---|---|---|
| | Diacel | | KH NeoChem | | Godavari | | Koken | | Target Spec | |
| Appearance | Clear | | Clear | | Clear | | Colorless | | Clear | |
| | Colorless | | Colorless | | Colorless | | Liquid | | Colorless | |
| | | | Liquid Almost | | Liquid | | | | Liquid | |
| | | | Odourless | | | | | | | |
| Color | 15 Pt—Co max. | | 10 APHA max. | | 10 Hazen max. | | | | 10 Hazen max. | |
| SG (20/20° C.) | 1.004 to 1.007 | | 1.004 to 1.007 | | 1.004 to 1.006 | | | | 1.004 to 1.006 | |
| Purity | | | 99.5% | min. | 99.50% | min. | 99.50% | min. | 99.50% | min. |
| Water Content | 0.5% | max. | 0.5% | max. | | | | | | |
| Distillation, | 203 | min. | 200 | min. | | | | | | |
| Initial boiling | | | | | | | | | | |
| point | | | | | | | | | | |
| Distillation, Dry | 209 | max. | 215 | max. | | | | | | |
| point | | | | | | | | | | |
| Free acid (as | 0.005% | max. | 0.005% | max. | 0.010% | max. | | | 0.005% | max. |
| acetic acid) | | | | | | | | | | |
| Distillate | | | 95% | min. | | | | | | |

| | | | | |
|---|---|---|---|---|
| pH | Neutral | | | |
| Odor | | Normal | Odorless | Odorless |

### Summary

Crude 1,3 Butanediol was added to a laboratory distillation system equipped with a reflux head and side stream take off. A vacuum of 20 mmHg was then applied. The system was brought to boil and held at total reflux for ˜15 minutes. At this point, the temperatures at the side stream and the column top were similar. The top reflux ratio was set to 1:1 to and light boilers removed for ˜45 minutes. After this time, the side stream was set to take off 1,3-butanediol at 1:1 and the reflux ratio was adjusted to ˜10:1. Under these conditions, the purity and the odor of the side stream were deemed unacceptable, and the side stream take off was stopped. Further lights were removed from the column top at a reflux ratio of ˜15:1 for the remainder of the day before being allowed to cool overnight. The column was restarted the next day under similar conditions. The side stream was set to take off 1,3-butanediol at 1:1 and the top reflux ratio was set to take off at 5:1. Small variations in the reflux ratio were made and the impact on odor and purity were observed. Generally, the odor and purity of the side stream improved with an increased reflux ratio. The rest of the distillation was run with a top reflux ratio of ˜5:1 and a side stream take off of 1:1. Towards the end of the distillation, the GC heavies in the side stream began to increase and knocked the purity of the side stream below the specification of a 99.5% minimum.

Crude bioBG was treated with a caustic solution (˜40 wt %-50 wt % NaOH), such that the final concentration of NaOH in solution was 0.5 wt %. This mixture of caustic solution in high odor bioBG was then reacted in a tank vessel or caustic reactor at 60° C. for 6 hours while stirring.

The reaction product was then fed into a distillation column where, the unreacted caustic and insoluble reaction product were removed as a bottoms waste; whereas, pure BG, referred to as a lights fraction, was distilled from the top of the column. Further distillation can be completed if required.

### Example 2: Sensory Analyses

Two different sensory analyses were performed. The first sensory analysis was performed upon receipt of the samples. The second sensory analysis was performed using nose clips and with a gold standard petroleum-derived 1,3-BG (DSP-650-5). TABLE 1 provides taste attributes to the off-flavor (DSP-650-3) sample, the neutral taste bioBG (DSP-650-4), and a petroleum-derived BG (DSP-650-5). Specifically, main attributes and other attributes were observed during the sensory analysis across the three samples. There was no noticeable smoky attribute since this is more of an aromatic (volatile) attribute than a taste attribute. However, the aroma was different according to the descriptors presented in TABLE 2 (aroma descriptors obtained from the first sensory analysis). This suggested that aromatic compounds are mainly causative of off notes in the off-flavor sample (DSP-650-3).

TABLE 1
Taste attributes

| | Main | |
|---|---|---|
| Samples | attribute | Other attributes |
| DSP-650-3 (off-flavor) | Bitterness | Metallic, astringent, |
| | | sour, sweet, medicinal |
| DSP-650-4 (neutral taste BG) | Bitterness | Metallic, astringent, |
| | | sweet, medicinal |
| DSP-650-5 (petroBG control) | Bitterness | Metallic, sour, sweet, |
| | | biting/sharp, medicinal |

TABLE 2
Aromatic attributes

| Samples | Main Aroma Attribute |
|---|---|
| DSP-650-3 (off-flavor) | Strong urea/urine-like, sweaty, |
| | antibiotic ointment (low) |
| DSP-650-4 (neutral taste BG) | Medicinal/antibiotic ointment, oily |

### Sensory Analysis of 3-Heptanone and 1-Octen-3-One

A rapid sensory analysis was performed for 3-heptanone (0.04±0.01 ng/g; 0.00004±0.00001 ppm) and 1-octen-3-one (0.015±0.002 ng/g; 0.000015±0.000002 ppm) compounds in water. The sensory was developed by five experienced panelists using nose clips to determine potential flavor attributes. The team found that compounds 3-heptanone and 2-ethyl-1-hexanol did not present any taste attribute; however, compound 1-octen-3-one presented a subtle taste attribute with strong earthy, mushroom, metallic retro nasal odor attributes. The descriptors for 1-octen-3-one were in alignment with the description provided by the panelists.

### Example 3: Identification of Non-Volatile Compounds in Samples

Off-flavor (DSP-650-3; equivalent to DSP-555-6 to DSP-555-12) and neutral taste bioBG (DSP-650-4; large scale of DSP-555-4) samples were provided for identification of non-volatile compounds that could negatively impact the bitter perception of the sample.

### Materials

Food-grade 100% ethanol was purchased from Decon Labs (King of Prussia, USA). Mobile-phase solvents and additives used for HPLC-MS analysis were Optima LC/MS grade obtained from Fisher Scientific (Fair Lawn, USA). Ethyl paraben was purchased from Sigma-Aldrich (St. Louis, MO). Standard at 95% of 1,3-butylene glycol (aka 1,3-Butanediol) was purchased from TCI America (Portland, USA). Water was purified using Barnstead Nanopure water system (Barnstead Nanopure Diamond water purification system, Thermo Scientific, Dubuque, IA). For sensory analyses, reverse osmosis water was used. DSP-650-3 (off-flavor sample) and DSP-650-4 (neutral taste bioBG) were stored at 10° C. upon receipt.

### Sensory Evaluation

Generally, samples were reconstituted in water. Each sample (5 mL) was presented to each of the panelists in 1-oz. black cups for evaluation. Samples were blind-coded and presented in a randomized order. To evaluate the samples, panelists were asked to swirl the sample in the mouth for 5-10 seconds and rate the bitterness intensity on a 5-point category scale from not present (0) to strong (4) before expectoration. Panelists used nose clips (Fisher Scientific) to prevent cross-modal interactions. Unsalted crackers and water were provided as palate cleansers between samples. There was a 1-minute break between samples to prevent any carry-over.

### Initial Sensory Screening

A sensory panel consisting of five panelists (ages 25-50; 3 females and 2 males) with sensory analysis experience evaluated the off-flavor (DSP-650-3) and neutral taste bioBG (DSP-650-4) samples in order to assess aroma attributes that differentiated the samples. The samples were diluted with water to 1% (v/v) and 4% (v/v) and presented to panelists in 4-oz. plastic souffle cups.

### Sensory Evaluation of SPE and Prep-HPLC Fractions

For fraction screenings, a sensory panel consisting of six panelists trained in evaluating bitterness (ages 25-50; 5 females and 1 male) utilized a 5-point category scale (0: not present, 1: threshold, 2: weak, 3: moderate, 4: strong) to evaluate the bitterness intensity of fractions obtained from both solid phase extraction (SPE) and preparative high performance liquid chromatography (prep-HPLC) fractionation. The sample dosage for each fraction was kept constant at an equivalent of original tasting concentration of 4% (v/v). The bitterness intensity score of each sample or fraction was then recorded as the average value of the group.

### High Resolution Mass Spectrometry (UHPLC/MS and MS/MS)

Analysis was conducted in a 1290 ultra-high performance liquid chromatography (UHPLC) system coupled with an Agilent 6546 LC/QTOF (LC/MS Quadrupole Time of Flight) mass spectrometer equipped with a Dual AJS ESI ion source, run in positive mode. The UHPLC system consisted of a degasser, a binary pumping system, an autosampler, a column heater, and a HSS T3 column (Acquity HSS T3 column, 1.8 m, 100 mm×2.1 mm). Mass spectrometric ionization conditions were as follows: gas temperature, 350° C.; gas flow, 10 L/min; nebulizer, 35 psi; sheath gas temperature, 375° C.; sheath gas flow, 11 L/min; capillary voltage, 4000 V; nozzle voltage, 500 V. References used for mass calibration were as follows: TFA anion, m/z 112.9856, and HP-0921 (+formate) anion, m/z 966.0007 were used as electrospray ionization (ESI) negative mode reference masses, purine, m/z 121.0509 and HP-0921, m/z 922.0098 were used as ESI positive mode reference masses. For scan mode, the range of m/z was 60-1700. Tentative identification was made using spectral matching against databases, such as the Human Metabolome Database (HMDB), to aid with the spectral identification process. The liquid chromatography (LC) gradient and solvent type were optimized to improve resolution and separation of compounds. Therefore, a flow rate of 0.5 mL/min was used with a binary mobile phase consisting of solvent (A) nanopure water with 0.1% formic acid and (B) Methanol with 0.1% formic acid. The gradient was as follows: 0-2 min, holding at 5% (B); 2-15.5 min, 5%-60% (B); 15.5-16.5 min, 60%-95% (B), 16.5-17.5 min, holding at 95% (B); 17.5-19.5 min, holding at 5% (B). The column heater was maintained at 40° C.

### Sample Fractionation

Both preparative High-Performance Liquid Chromatography (Prep-HPLC) separation and solid-phase extraction (SPE) separation were carried out separately to fractionate the off-flavor sample (DSP-650-3).

### Preparative High-Performance Liquid Chromatography (Prep-HPLC)

DSP-650-3 was further fractionated to locate the bitter tasting compounds. For this purpose, a prep-HPLC system containing a binary pumping system (Waters 2545), a sample manager and automated sample injector and fraction collector (Waters 2767), and a PDA detector (Waters 2489) connected to a triple quadrupole mass spectrometer (Waters Quattro micro) was used for first dimension fractionation. The mass spectrometer parameters used were as follows: capillary voltage: 2.5 kV for negative mode and 3.0 kV for positive mode, cone voltage: 30 V, cone gas flow: 60 L/h, source temperature: 120° C., desolvation gas temperature: 400° C., drying gas: 1200 L, desolvation gas at 650 L/h, quadrupole scan range of m/z 100-1200.

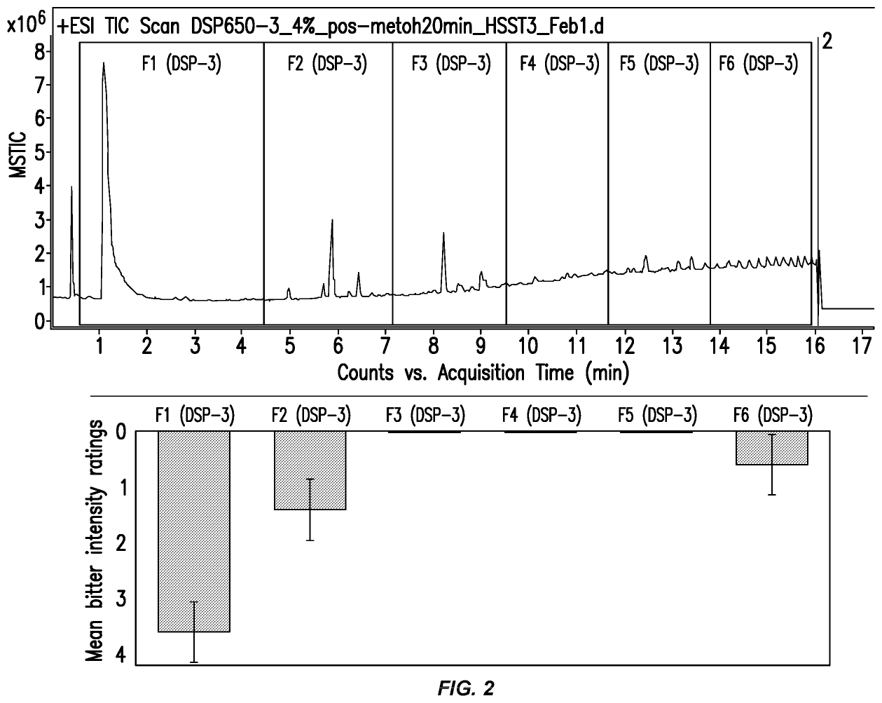

For the first-dimension fractionation, 4 mL of DSP-650-3 was dissolved in 12 mL water and filtered through a 0.45 m Millipore Millex-HV hydrophilic polyvinylidene fluoride (PVDF) membrane filter (Millipore Sigma, USA) before injection into the HPLC system. Aliquots of the isolate were injected onto a preparative C18 column (5 μm, 50 mm×50 mm, XBridge BEH C18, Waters Co.) maintained at 25° C. Chromatography was performed using a binary solvent system of water (A) and methanol (B). The elution gradient was as follows: 0-2 min, holding at 5% (B); 2-12 min, 5%-60% (B); 12-13 min, 60%-95% (B), 13-14 min, holding at 95% (B); 14-16 min, holding at 5% (B). The flow rate was 100 mL/min. The effluent was collected as 6 subfractions using a fraction collector. The corresponding fractions (F1-F6) obtained from several runs were pooled and evaporated (FIG. 2; upper panel). The fractions were dissolved in a volume equivalent to the original tasting concentration of 4% (v/v) and evaluated by a trained sensory panel for bitterness intensity (FIG. 2; lower panel) on a 5-point scale. All of the fractions were also further analyzed by the high-resolution LC/MS system.

### Solid Phase Extraction (SPE)

DSP-650-3 (4 mL in 20 mL water) was fractionated using the 6 g Oasis HLB cartridge (Waters Co, Milford, MA). For this step, the cartridge was first pre-conditioned with 20 mL of 95% ethanol/water, followed by 20 mL of 5% ethanol/water. The sample was then loaded on the cartridge and washed with 40 mL of 5% ethanol/water to yield aqueous fraction (F1). The sample was then eluted using different ethanol/water ratios namely 15% ethanol/water (F2), 30% ethanol/water (F3), 45% ethanol/water (F4), 60% ethanol/water (F5), 75% ethanol/water (F6), and 95% ethanol/water (F7). The fractions were pooled individually and removed from solvent. All of the samples were then evaluated for sensory evaluation. SPE was performed in duplicates.

### Results of Initial Sensory Screening

Both DSP-650-3 (off-flavor) and DSP-650-4 (neutral taste bioBG; large scale of DSP-555-4) samples were diluted with water to a concentration of both 1% and 4% and were presented to panelists for taste screening. An initial sensory evaluation revealed that at a concentration of 4%, bitterness was perceivable in both samples, with the off-flavor sample (DSP-650-3) rated as more bitter than the neutral taste bioBG sample (DSP-650-4).

### Results of First Dimension Fractionation Using Preparative-HPLC

Sensory-guided fractionation using prep LC-fractionation was carried out to identify compounds responsible for the bitter taste in the off-flavor sample (DSP-650-3). To isolate the key bitter compounds, first-dimension fractionation was completed (FIG. 2). First, DSP-650-3 was separated by a C18 column to yield 6 different subfractions (F1-F6) (FIG. 2). Sensory evaluation by trained panelists revealed that F1 was found to have a moderate to strong bitter intensity (scale: 3-4), the highest bitter intensity among all of the subfractions. The remaining subfractions F2-F6 were found to have at most threshold bitter intensity (scale: 1). Therefore, F1 was submitted for UHPLC/MS analysis.

### Results of Fractionation Using SPE

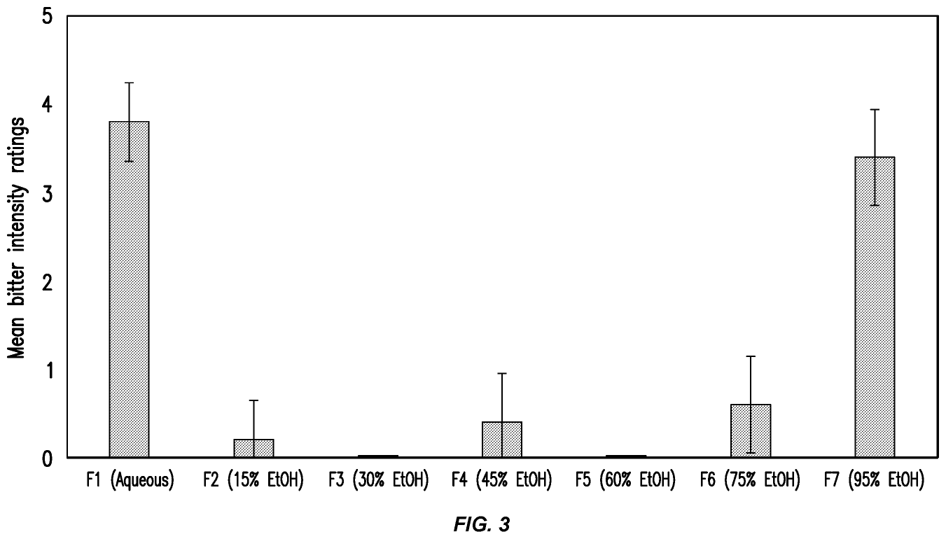

SPE was used as a complementary tool for the separation of the off-flavor sample (DSP-650-3). From the SPE fractionation, 7 different subfractions (F1-F7) were obtained. Sensory panelists trained in evaluating bitterness evaluated the subfraction samples (FIG. 3). Sensory evaluation revealed that F1 (aqueous) and F7 (95% ethanol/water (v/v) elute) have moderate to strong bitter intensity. The remaining fractions were found to have at most threshold intensity. Therefore, F1 and F7 were submitted for UHPLC/MS analysis.

### Results of Analysis of Fractions Using UHPLC/MS System

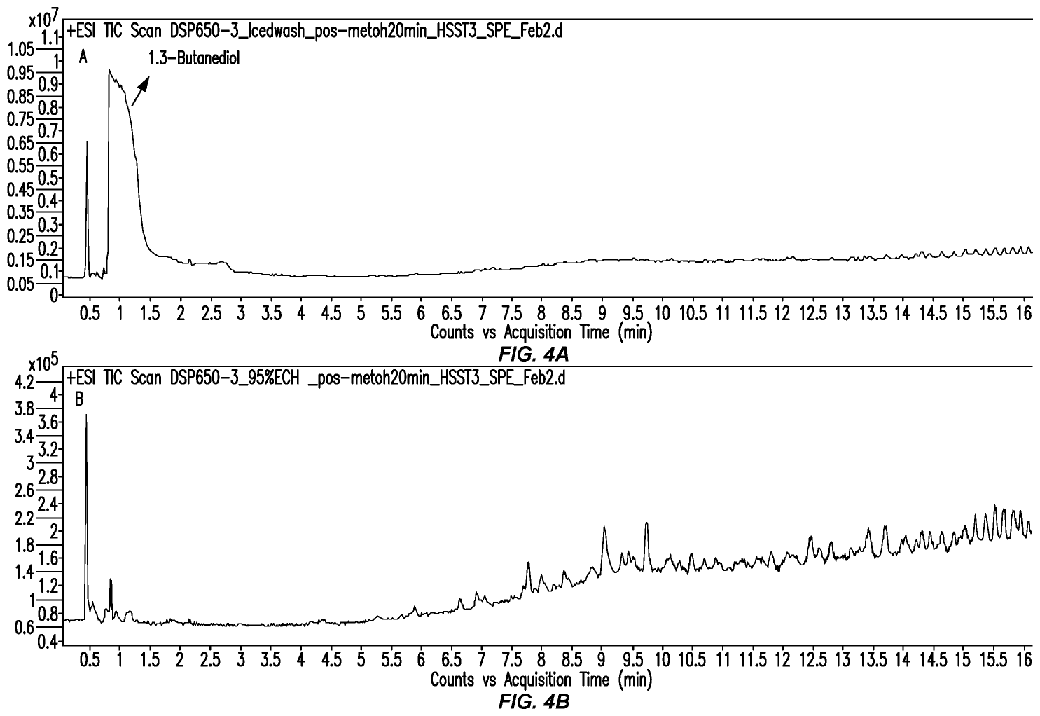

Analysis of subfraction F1 obtained from the preparative HPLC fractionation revealed that this fraction contained the majority of solvent peak (1,3-butylene glycol, aka 1,3-Butanediol) (FIG. 4A; upper panel). Similarly, further analysis of F1 obtained from the SPE fractionation also revealed high intensity of 1,3-butylene glycol in the chromatogram. This indicates that the results obtained from the preparative HPLC fractionation and SPE fractionation were consistent and further indicates that 1,3-butylene glycol which is the solvent carrier can be the source of bitterness in the sample. To further determine the bitter taste impact of 1,3-butylene glycol, further sensory and UHPLC analysis was conducted. Regarding fraction F7 obtained from SPE fractionation, this fraction displayed low ionizable peaks and complexity as shown in FIG. 4B (lower panel).

### Results of Analysis of 1,3-Butylene Glycol by Sensory Evaluation and UHPLC/MS

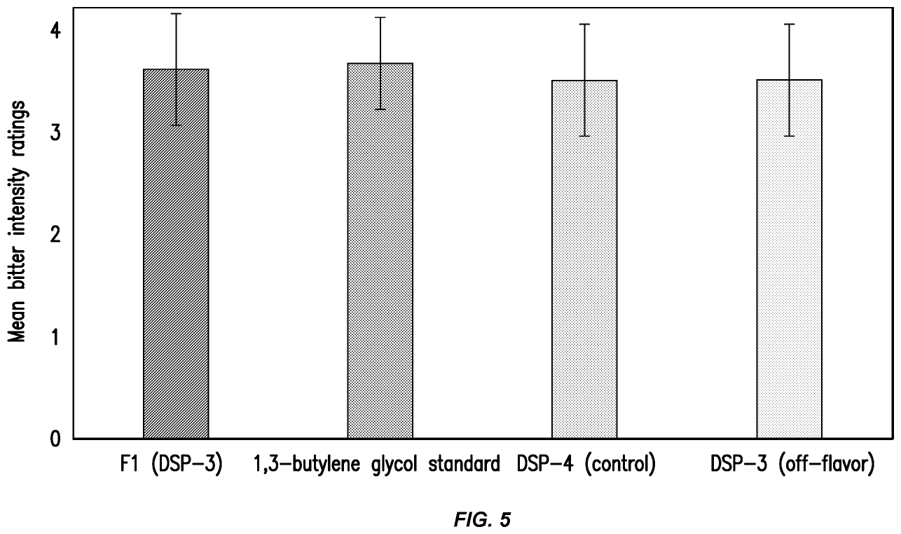

Another sensory evaluation was conducted to analyze the bitterness intensity of subfraction F1 and how it compared to the standard of 1,3-butylene glycol, off-flavor (DSP-650-3), and neutral taste bioBG (DSP-650-4). The standard solvent, DSP-650-3, and DSP-650-4 were evaluated at a concentration of 4% (v/v), and subfraction F1 was reconstituted with water to mimic the tasting concentration of 4% (v/v) as well. From the sensory evaluation (FIG. 5), the bitterness intensity of the 1,3-butylene glycol standard was found to be very similar to subfraction F1 of DSP-650-3 and both the off-flavor sample (DSP-650-3) and neutral taste bioBG sample (DSP-650-4) at moderate to strong bitter intensity, indicating that 1,3-butylene glycol contributes to the bitter perception in the sample. Further analysis of the UHPLC/MS-QToF on all of the samples showed similar a peak area of 1,3-butylene glycol (TABLE 3), indicating that the amount of 1,3-butylene glycol in the samples were comparable and therefore aligned with the sensory results in which all samples were rated to have comparable bitter intensity.

However, it was surprising that the bitter intensity of the off-flavor sample and neutral taste bioBG sample were similar at moderate to strong intensity since initial sensory screening showed that the neutral taste bioBG sample was rated as a less bitter sample. Because of this, the sensory evaluation was repeated in duplicates. From the second sensory evaluation, panelists rated both off-flavor (DSP-650-3) and neutral taste bioBG (DSP-650-4) samples to have similar bitter intensity at moderate to strong intensity, showing reproducibility and consistency of the panelists. From both sessions, panelists indicated that the off-flavor sample (DSP-650-3) has other taste attributes of biting, irritating bitterness which was not present in the neutral taste bioBG sample (DSP-650-4). From the sensory evaluation, discrepancy in the sensory evaluation results between the initial screening to the latest sensory evaluation could also be due to the oxidation or degradation in the DSP-650-4 neutral taste bioBG sample, which would have increased the bitterness intensity of the sample. For this sensory evaluation, off-flavor (DSP-650-3) and neutral taste bioBG (DSP-650-4) samples have been stored in a refrigerator at 10° C. for approximately 3 weeks.

### Relative Quantification Results

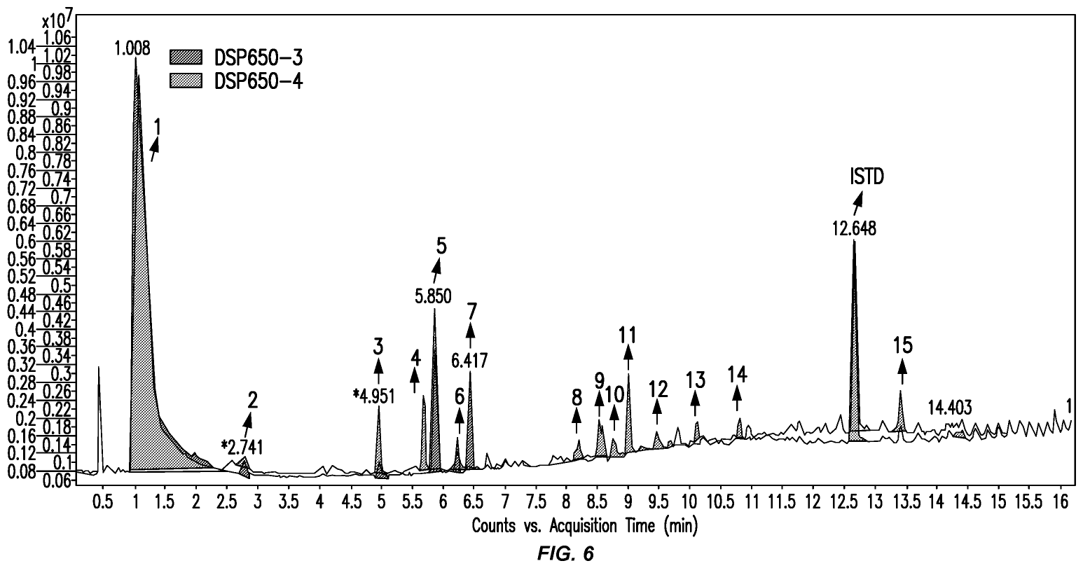

Relative quantification was performed on a 1290 UHPLC system coupled to a QTOF mass spectrometer as stated above (see, e.g., “High Resolution Mass Spectrometry (UHPLC/MS and MS/MS)”). Both samples, off-flavor (DSP-650-3) and neutral taste bioBG (DSP-650-4), (n=3) were spiked with ethyl para-hydroxybenzoate as internal standard (ISTD) for further quantification. Off-Flavor and Neutral Taste bioBG columns are the peak area associated with different compounds. In general, all of the detected peaks in the off-flavor sample were in major relative abundance as compared to the neutral bioBG sample, which ranged from 16.34%-553.36% as reported in TABLE 3 and FIG. 6. Nine peaks detected in the off-flavor sample were found to be in common with the neutral bioBG sample (see, Peak #s 1-3, 5-7, 13-14, and 15). The relative percent increase in compound concentration for the uncommon peaks (see, Peak #s: 4 and 8-12) of the off-flavor sample compared to those of the neutral taste bioBG sample ranged from 18734.47%-70646.44%.

TABLE 3
Relative concentration of non-volatile compounds in samples

| | | | | | | Relative |
|---|---|---|---|---|---|---|
| | | | | Neutral Taste | | concentration: |
| | | | Off-Flavor | bioBG | | off-flavor vs |
| Peak | RT | Relative RT | (DSP-650-3) | (DSP-650-4) | | neutral taste |
| # | (mins) | (mins) | (ppm) | (ppm) | Compound | bioBG |
| 1 | 1.005 | 1 | 8.789 | 7.554 | 1,3-BG | 16.34% |
| 2 | 2.760 | 2.733 | 0.142 | 0.022 | | 553.36% |
| 3 | 4.948 | 4.899 | 0.256 | 0.040 | | 548.06% |
| 4 | 5.67 | 5.614 | 0.307 | 0.000 | | 67184.36% |
| 5 | 5.847 | 5.789 | 0.779 | 0.536 | | 45.22% |
| 6 | 6.225 | 6.163 | 0.140 | 0.078 | | 79.18% |
| 7 | 6.425 | 6.361 | 0.420 | 0.338 | | 23.97% |
| 8 | 8.202 | 8.121 | 0.135 | 0.000 | | 29437.6% |
| 9 | 8.513 | 8.429 | 0.221 | 0.000 | | 48323.54% |
| 10 | 8.758 | 8.671 | 0.086 | 0.000 | | 18734.47% |
| 11 | 9.002 | 8.913 | 0.323 | 0.000 | | 70646.44% |
| 12 | 9.435 | 9.341 | 0.107 | 0.000 | | 23266.33% |
| 13 | 10.102 | 10.002 | 0.145 | 0.041 | | 251.07% |
| 14 | 10.802 | 10.695 | 0.090 | 0.060 | | 49.25% |
| 15 | 13.413 | 13.280 | 0.207 | 0.063 | | 229.61% |

### UHPLC/MS/MS Analysis Results

The MS/MS spectrum was obtained by a collision energy setting of 10 V, 30 V, and 60 V for off-flavor (DSP-650-3), neutral taste bioBG (DSP-650-4), and 1,3-butylene glycol (1,3-BG) samples. As shown in TABLE 4, the peaks and their mass-to-charge ratio (m/z) and intensity of the off-flavor sample were compared to the Human Metabolome Database (HMBD).

TABLE 4
MS/MS fragments of off-flavor sample at collision energy of 10 V.

| | Relative | Off- | Neutral Taste | | |
|---|---|---|---|---|---|
| RT | RT | Flavor | bioBG | | |
| (min) | (mins) | (DSP-650-3) | (DSP-650-4) | MS/MS (m/z) | Compound |
| 0.996* | 1 | X | X | 91.0756; 73.0648; 55.0541 | 1,3-BG* |
| 2.658 | 2.679 | X | X | 191.0737; 172.9775; 141.9585; | |
| | | | | 87.0804; 69.0700 | |
| 4.801 | 4.840 | X | X | 127.0774; 109.0650; 81.0699 | |
| 5.493 | 5.537 | X | | 204.1591; 159.1012; 141.9584; | |
| | | | | 89.0593; 60.0803 | |
| 5.683 | 5.729 | X | X | 143.1070; 103.0756; 89.0599; | |
| | | | | 71.0493 | |
| 6.047** | 6.096 | X | X | 206.1753; 143.1070; 86.0966; | |
| | | | | 60.0807 | |
| 6.237** | 6.287 | X | X | 206.1757; 161.1178; 143.1070; | |
| | | | | 89.0601; 71.0494 | |
| 7.978 | 8.042 | X | | 103.0751; 89.0595; 71.0489; | |
| | | | | 60.0804 | |
| 8.343 | 8.410 | X | | 310.2595; 296.2439; 141.9597; | |
| | | | | 100.1129; 86.0973 | |
| 8.817 | 8.888 | X | | 310.2585; 296.2432; 161.1171; | |
| | | | | 86.0962; 60.0805 | |
| 9.275 | 9.350 | X | | 167.1067; 141.9587; 86.0964; | |
| | | | | 60.0806 | |
| 13.229 | 13.335 | X | X | 199.1331; 141.9586; 100.1120; | |
| | | | | 86.0964 | |

X Peaks in common
*confirmed with pure standard
**impurities or isomeric forms of 1,3-butylene glycol confirmed with pure standard

### Example 4: Qualitative/Quantitative Testing on Neutral Taste bioBG Sample

A comparative analysis of neutral taste bioBG samples (DSP-723-1; DSP-723-2; DSP-723-3; DSP-723-4; DSP-723-5) was performed by both GC/MS and headspace GC/MS, and a comparative analysis was also performed by GC/MS of samples of food grade 1,3-butylene glycol to the neutral taste bioBG samples in order to identify compounds that produced an off-taste or undesirable taste.

GC/MS analysis resulted in the detection of an unknown peak that was present in all of the samples and could not be identified with a high degree of certainty, but that was more prevalent in sample DSP-723-3 than in the other samples (DSP-723-1; DSP-723-2; DSP-723-4; DSP-723-5). Testing by headspace GC/MS indicated that toluene was present in all of the samples, which was believed to be the compound contributing to the off-taste characteristics that had been previously observed.

### Instrumentation

Gas Chromatography/Mass Spectrometry (GC/MS): GC/MS testing was used for the analysis of samples along multiple dimensions of chemical properties, providing specific identification of the different compounds separated during the GC analysis. The gas chromatograph separated a complex mixture into its individual components and delivered each one to the mass spectrometer. This analysis generated a chromatogram consisting of different peaks, one for each component of a mixture. The area of each peak was used to measure quantity. GC/MS analysis was used both for qualitative and quantitative determinations of chemical composition.

Solid-Phase Microextraction (SPME): SPME is a solid phase extraction sampling technique that involves the use of a fiber coated with an extracting phase, which is capable of extracting analytes from sample headspace. The fibers used included: Divinylbenzene/Carboxen/Polydimethylsiloxane (DVB/CAR/PDMS) fibers. SPME, paired with an analytical technique, was used for both identification and quantitation of sample analytes.

### Analytical Testing

GC/MS Method Designed for Analysis of Neutral Taste bioBG Samples

Sample solutions (DSP-723-1; DSP-723-2; DSP-723-3; DSP-723-4; DSP-723-5) and a standard solution of 1,3-butylene glycol (Acros Organics, 99%) were prepared by pipetting 0.1 mL of each into individual 10-mL volumetric flasks and then each was diluted to volume with methanol. The samples were mixed well and analyzed via GC/MS using the following parameters.

### Instrument Conditions for Analysis:

Instrument: Agilent 6890N GC with Agilent 5973 Mass Selective Detector (MSD)
Column: DB-Waxetr 30 m×0.25 mm, 0.25 m film

TABLE 5
Temperature Program

| Ramp Rate | Final Temperature | Hold Time |
|---|---|---|
| (° C./min) | (° C.) | (min) |
| 0 | 40 | 3 |
| 10 | 80 | 0 |
| 5 | 130 | 2 |
| 10 | 170 | 0 |
| 25 | 240 | 0 |

Injection Volume: 1.0 L
Injection Mode: Split, 2:1
Inlet Temperature: 230° C.
Flow Rate: 2.8 mL/min
Detection Mode: Scan (32-800 m/z)
Detector Temperature: 240° C.
   Headspace GC/MS with SPME Method Designed for Analysis of Neutral Taste bioBG Samples

Neutral Taste bioBG samples (DSP-723-1; DSP-723-2; DSP-723-3; DSP-723-4; DSP-723-5) and a standard of 1,3-butylene glycol were prepared by pipetting 1 mL of each into individual headspace vials. Each headspace vial was heated at 80° C. for 45 minutes, and then a SPME fiber was used to collect volatiles for analysis by exposing the fiber to the headspace of the vial for two minutes. This was subsequently injected onto a GC/MS and tested using the following parameters.

### Instrument Conditions for Analysis:

Instrument: Agilent 6890N GC with Agilent 5973 MSD
Column: DB-Waxetr 30 m×0.25 mm, 0.25 m film
Temperature Program: See, TABLE 5.
Injection Volume: 1.0 L
Injection Mode: Split, 2:1
Inlet Temperature: 230° C.
Flow Rate: 2.8 mL/min
Detection Mode: Scan (30-300 m/z)
Detector Temperature: 240° C.
   Headspace GC/MS with SPME Method Designed for Analysis of Neutral Taste bioBG Samples

Samples (DSP-723-1; DSP-723-2; DSP-723-3; DSP-723-4; DSP-723-5), a standard of 1,3-butylene glycol, and a standard of toluene (VWR, ACS grade) were prepared by pipetting 1 mL of each into individual headspace vials. Each headspace vial was heated at 80° C. for 45 minutes, and then a SPME fiber was used to collect volatiles for analysis by exposing the fiber to the headspace of the vial for two minutes (seven seconds for toluene). This was subsequently injected onto a GC/MS and tested using the following parameters.

### Instrument Conditions for Analysis:

Instrument: Agilent 6890N GC with Agilent 5973 MSD
Column: DB-5MS 30 m×0.25 mm, 0.25 m film

TABLE 6
Temperature Program

| Ramp Rate | Final Temperature | Hold Time |
|---|---|---|
| (° C./min) | (° C.) | (min) |
| 0 | 35 | 1 |
| 5 | 180 | 1 |
| 25 | 300 | 2 |

Injection Volume: mSPME: 2 mm
Injection Mode: Spitless
Inlet Temperature: 250° C.
Flow Rate: 2.8 mL/min
Detection Mode: Scan (30-300 m/z)
Detector Temperature: 280° C.

### Results of Analytical Testing

### Analysis by GC/MS: 1,3-Butylene Glycol Method

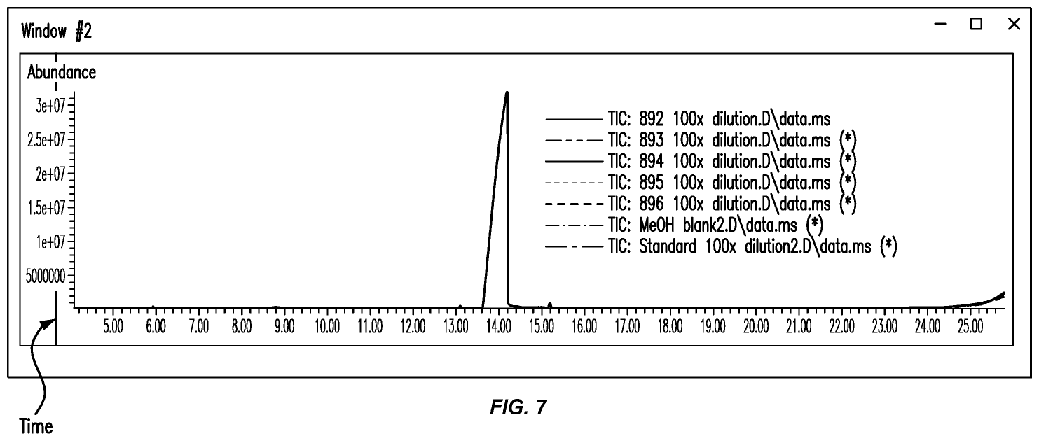

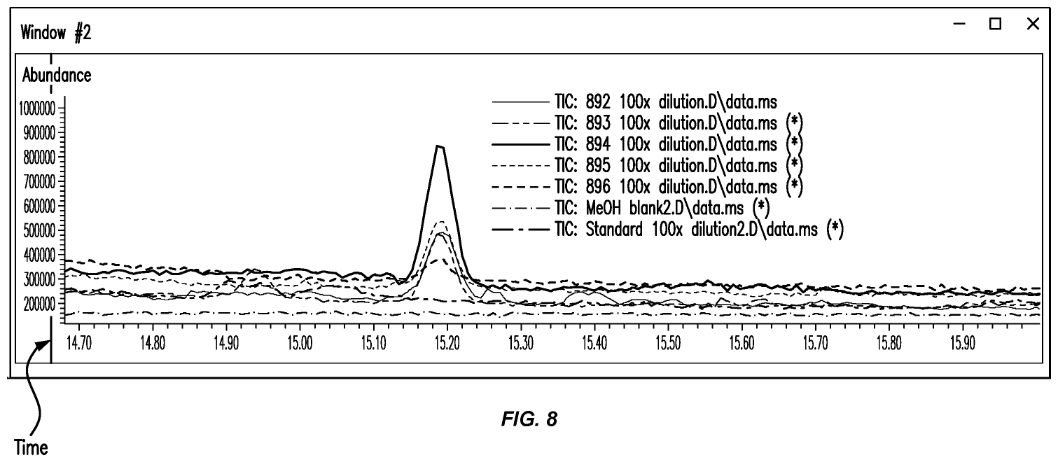

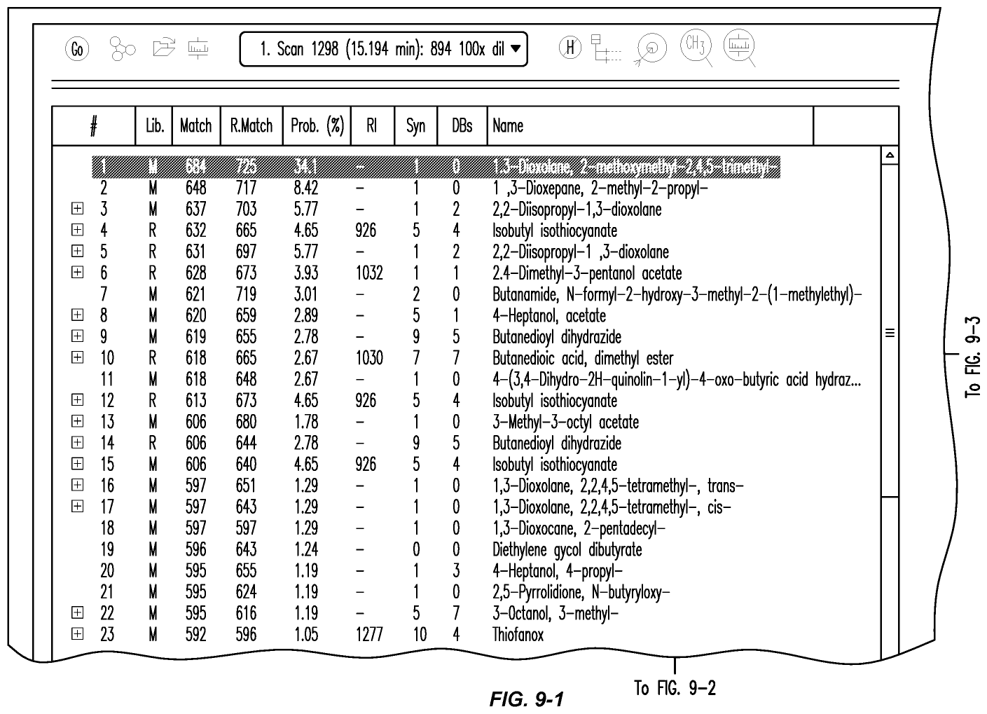

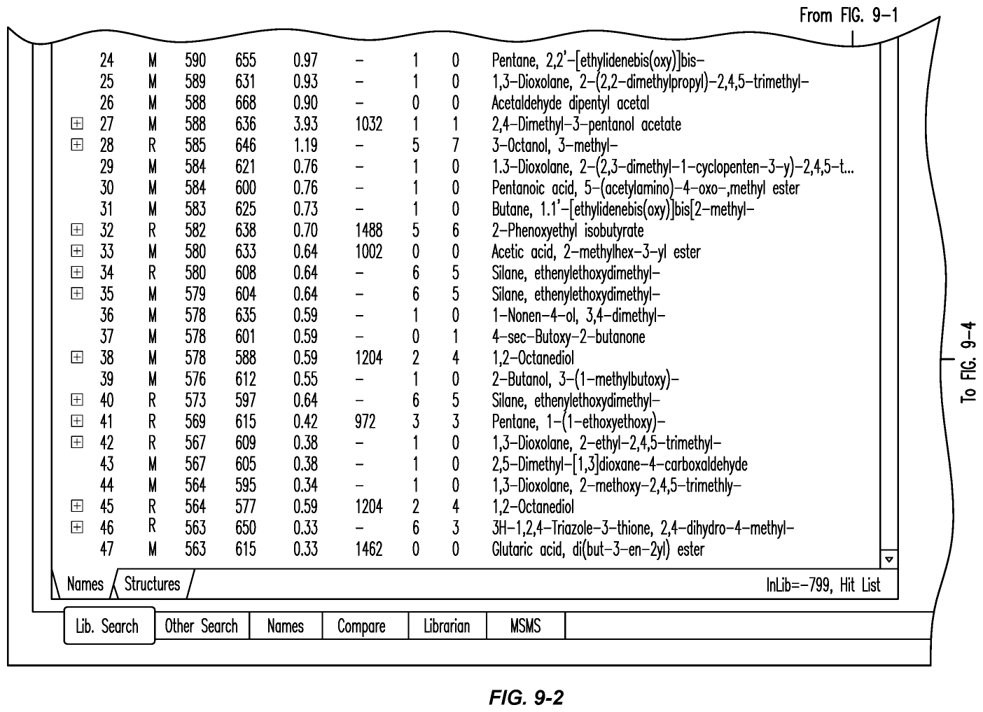

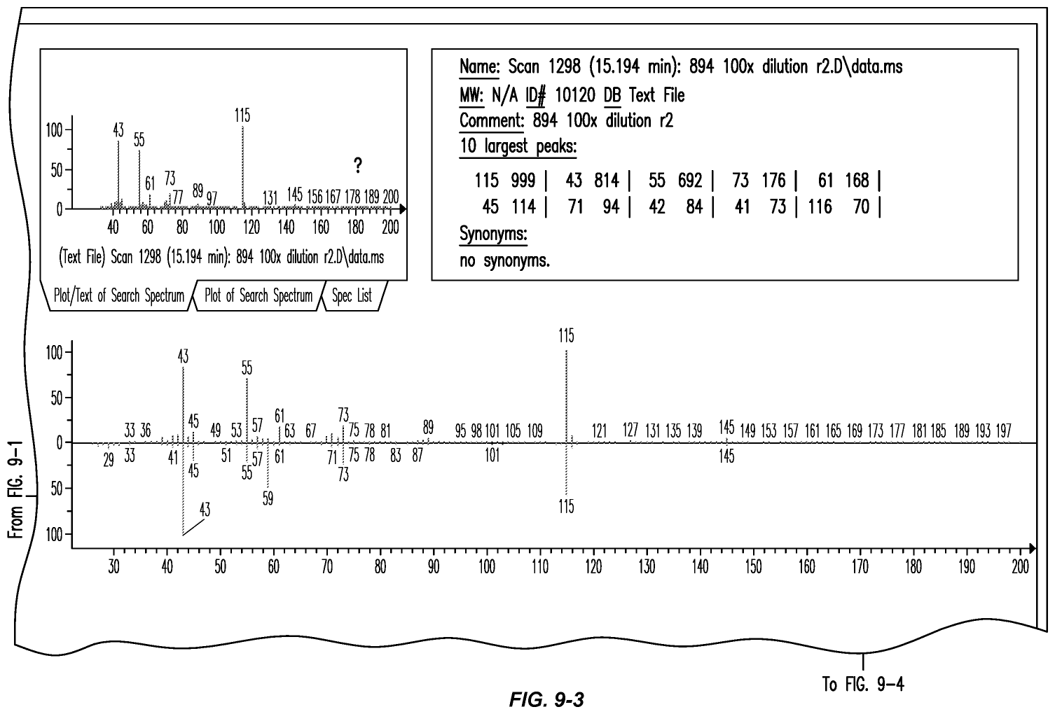

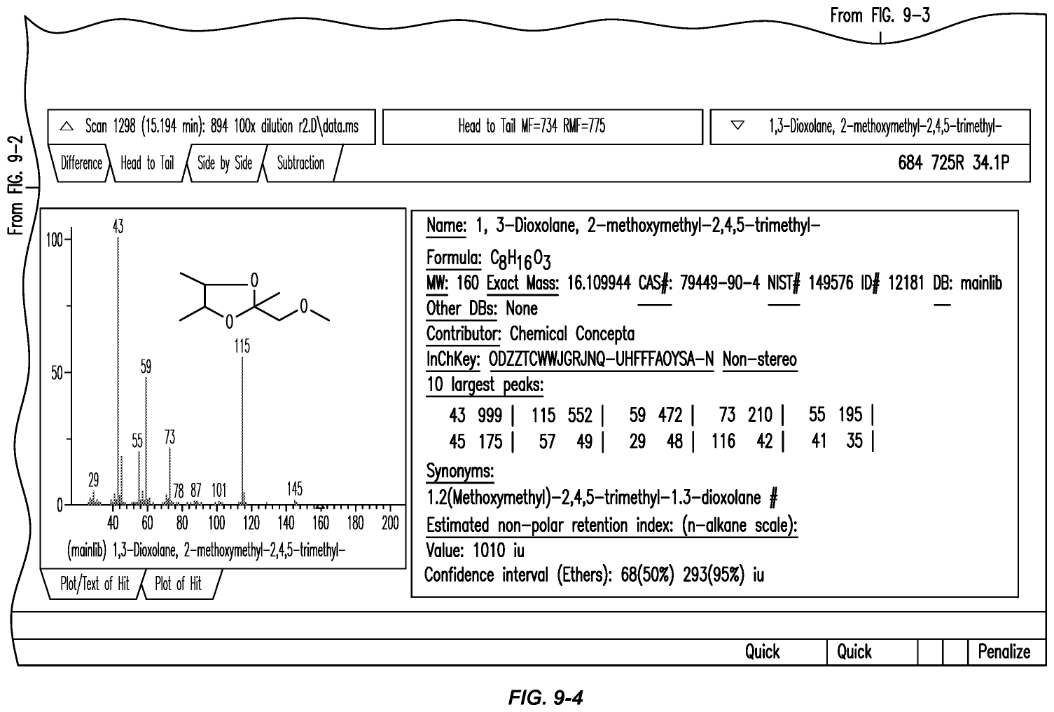

Chromatograms of the sample solutions and the 1,3-butylene glycol standard solution were compared against each other, and a peak with a retention time of 15.20 mins was observed for the samples. In particular, sample DSP-723-3 had a notably more intense peak than when compared with the other samples. No peak was detected at this retention time in the 1,3-butylene glycol standard. Using a NIST library match database, this peak was identified as being 1,3-dioxolane, 2-methoxymethyl-2,4,5-trimethyl (also known as 2-methoxymethyl-2,4,5-trimethyl-1,3-dioxolane). The results for this testing can be seen in FIGS. 7-9. This compound (2-methoxymethyl-2,4,5-trimethyl-1,3-dioxolane) can be a contributor to off-flavor in a neutral odor bioBG described here.

Analysis by Headspace GC/MS with SPME: 1,3-Butylene Glycol Method

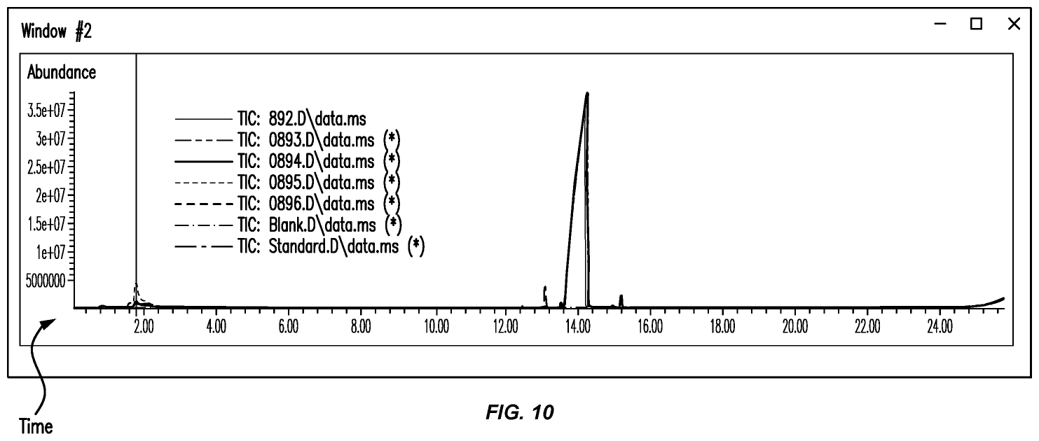

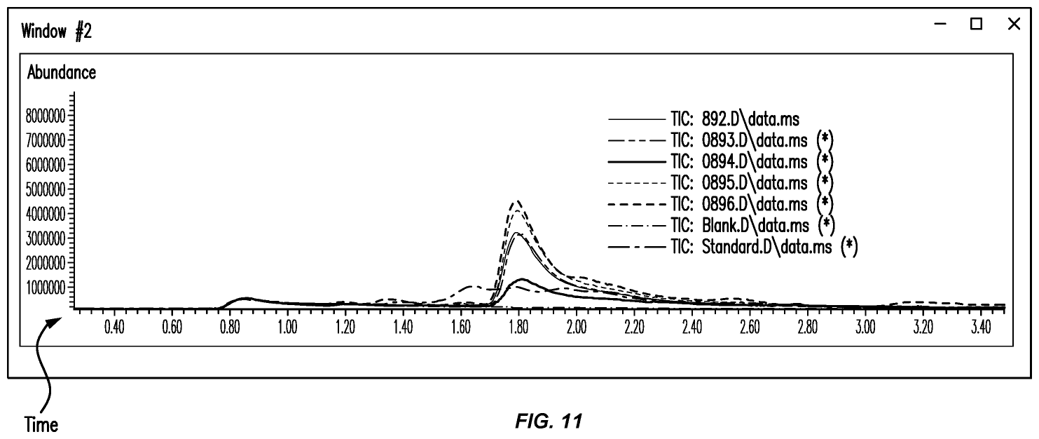

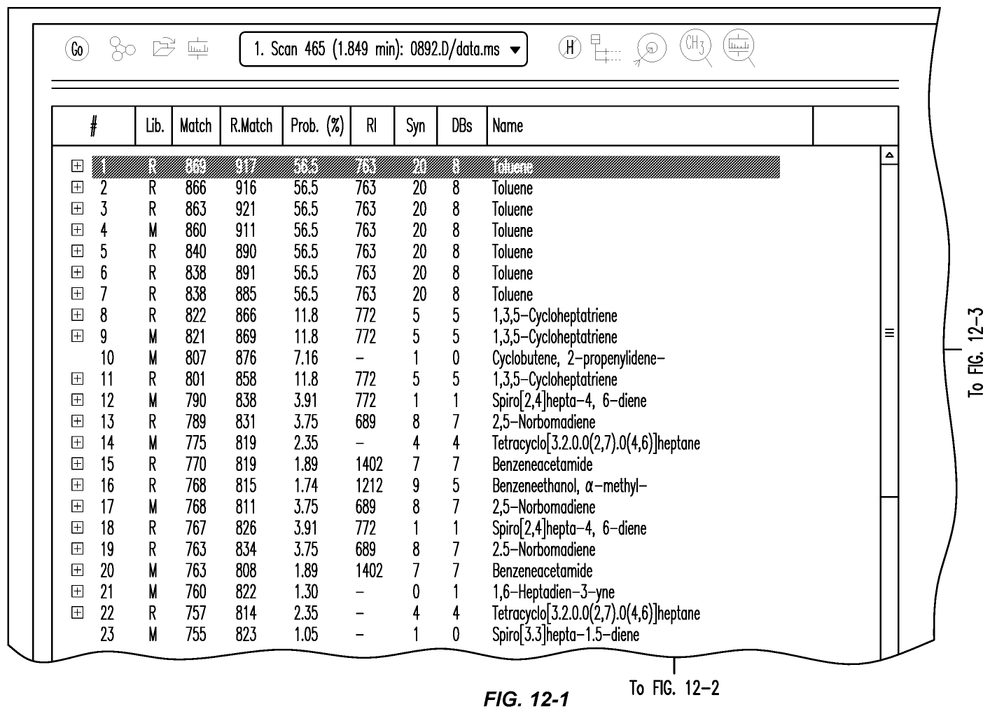

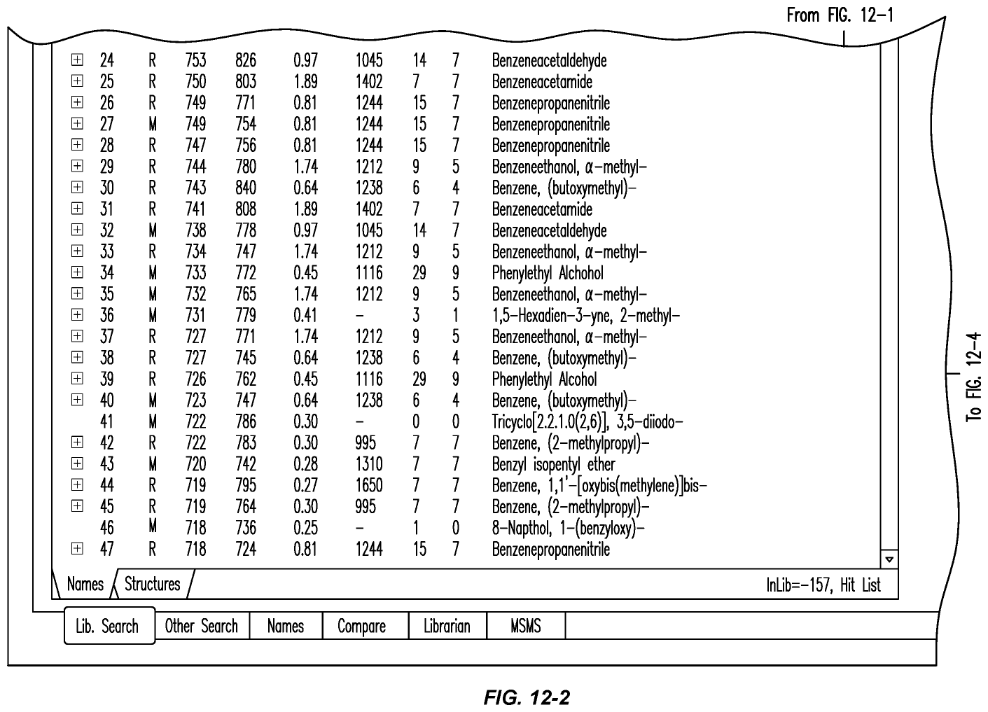

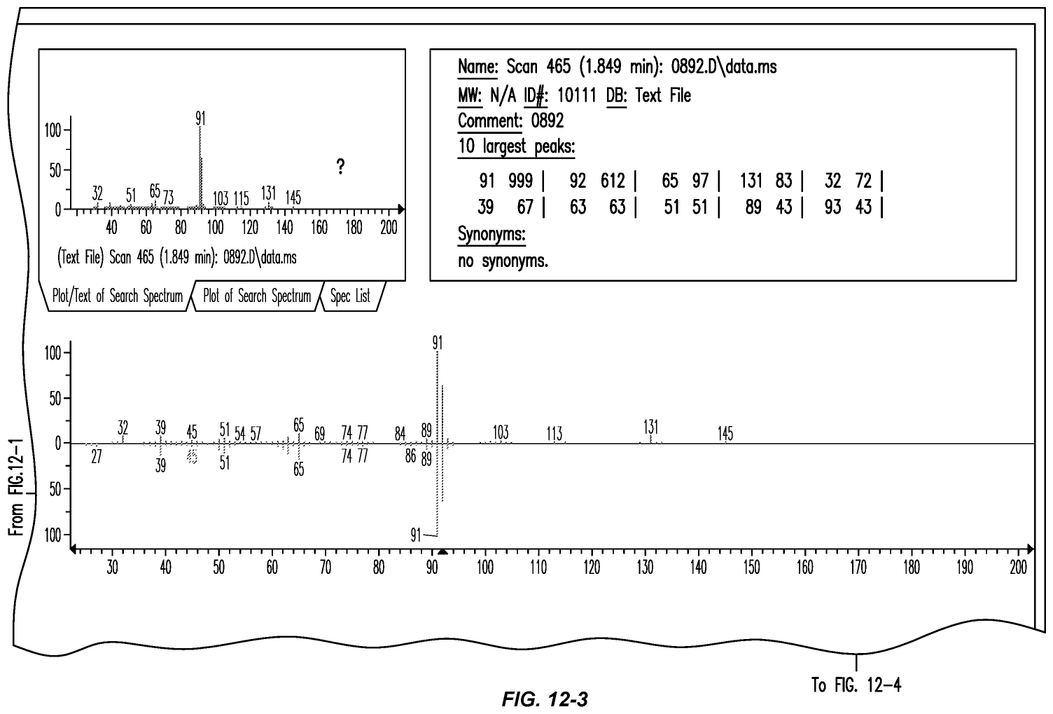

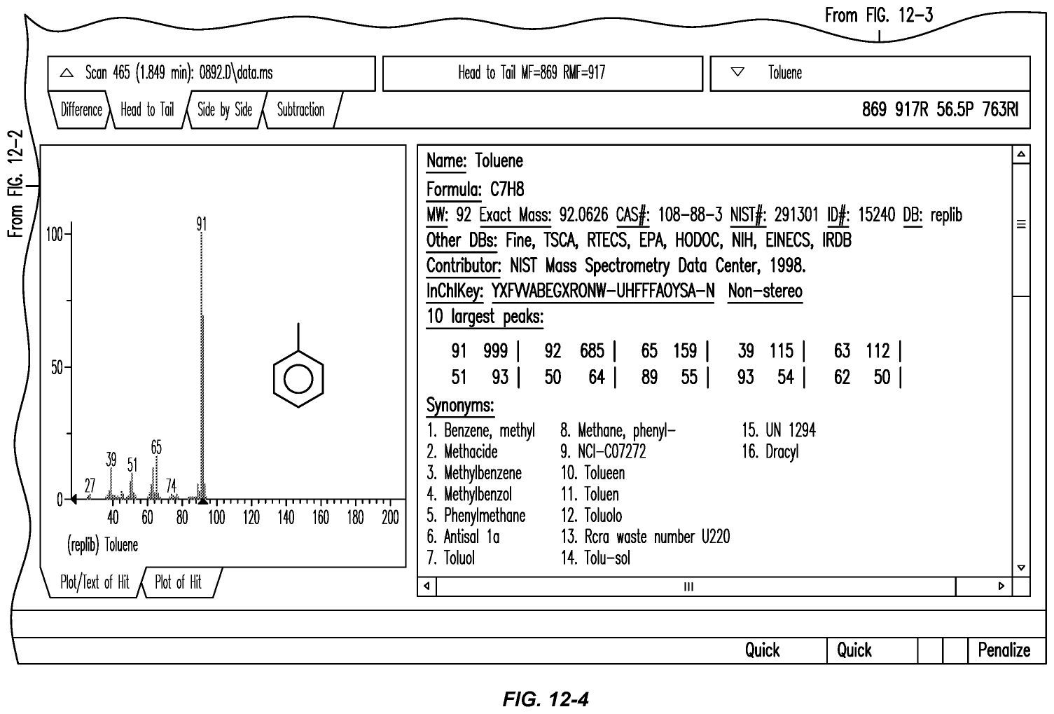

Chromatograms of the samples and the 1,3-butylene glycol standard were compared against each other, and a peak with a retention time of 1.80 mins was observed in all of the samples, but was not present in the 1,3-butylene glycol standard. Using a NIST library match database, this peak was identified as being toluene. A 1,3-butylene glycol peak was also observed in all samples and in the 1,3-butylene glycol standard at a retention time of 13.7 mins. The results for this testing can be seen in FIGS. 10-12. This compound (toluene) can be a contributor to off-flavor in a neutral odor bioBG described here.

Analysis by Headspace GC/MS with SPME: Volatiles Method

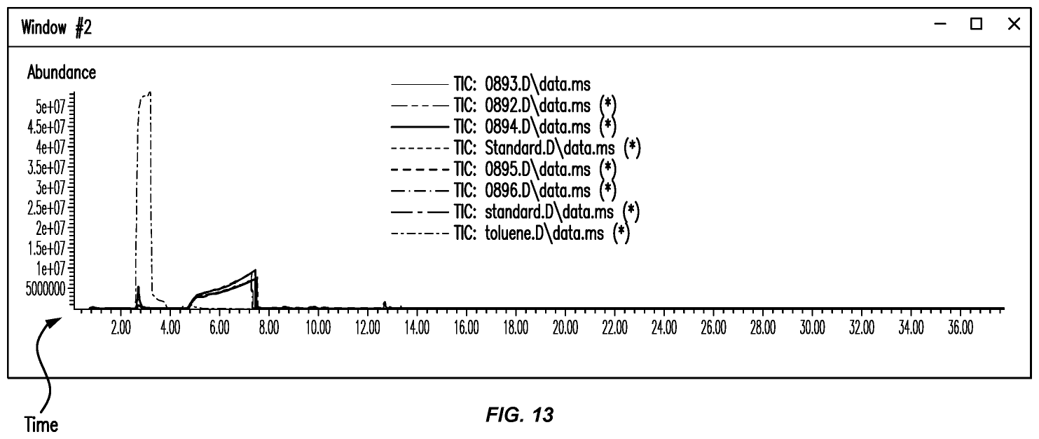

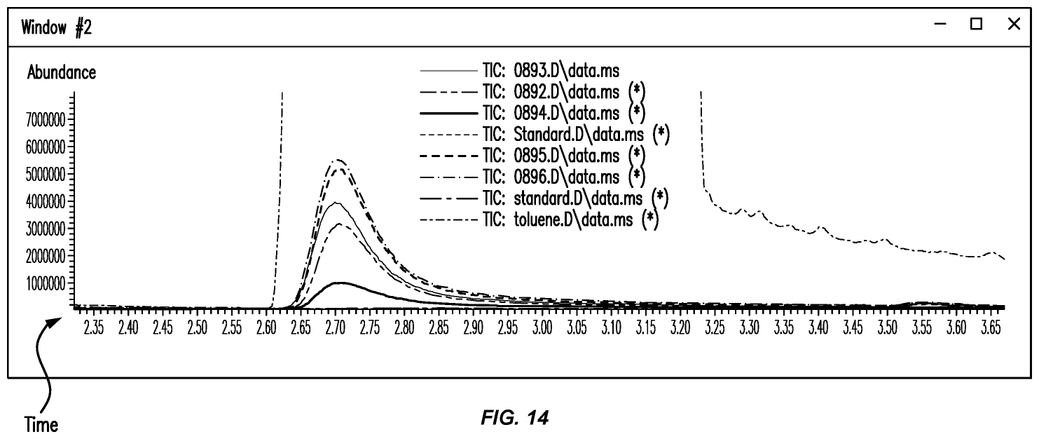

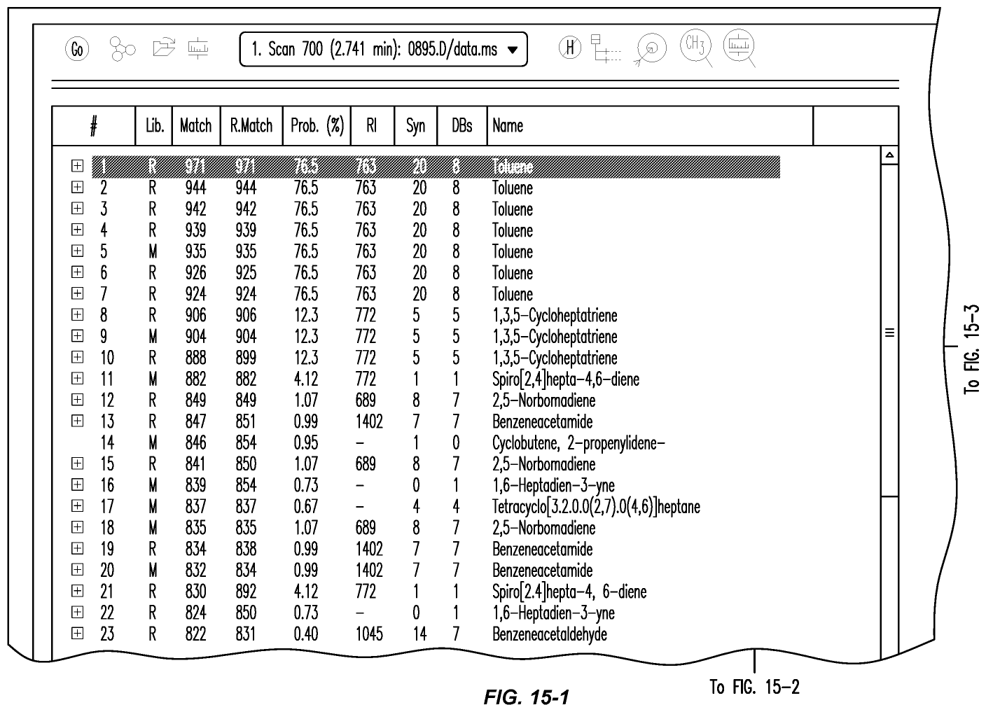

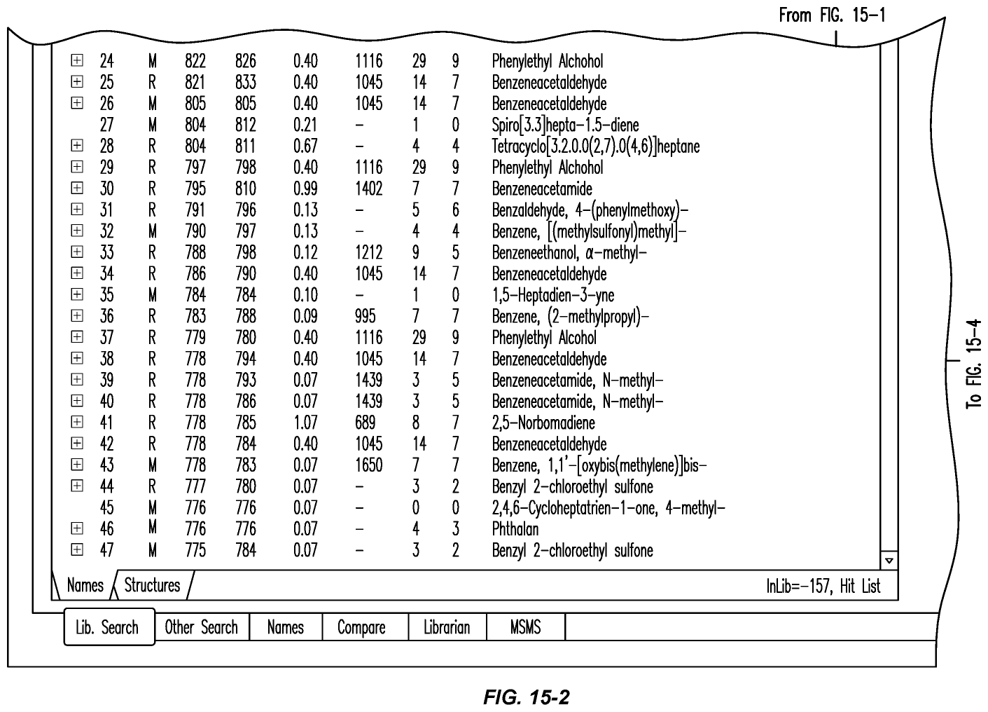

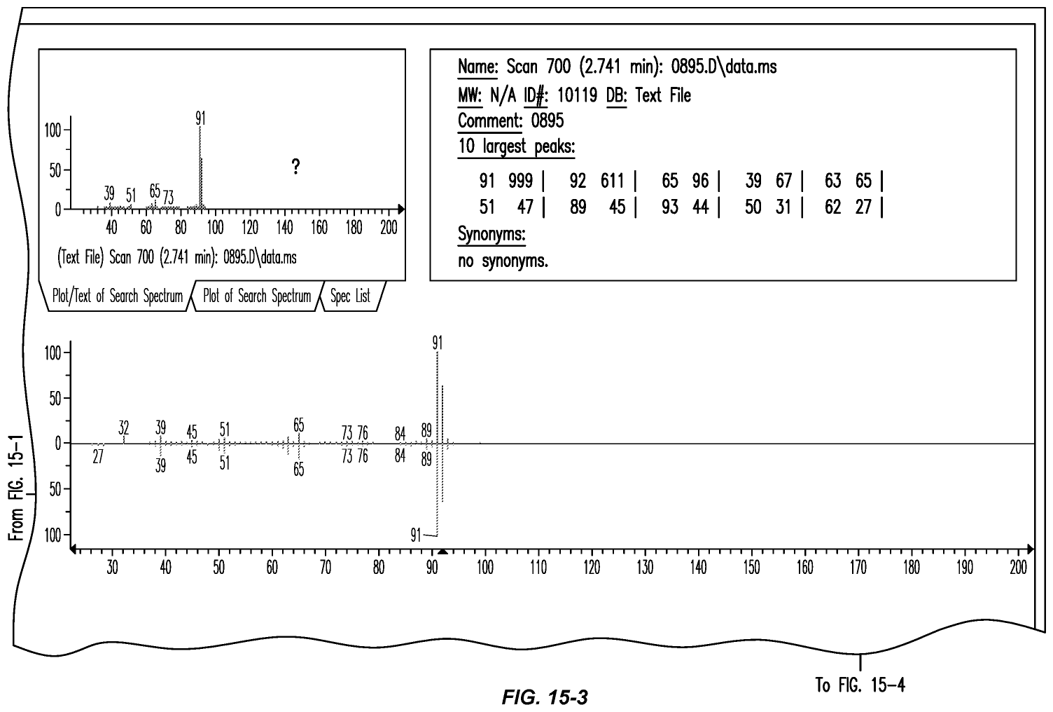

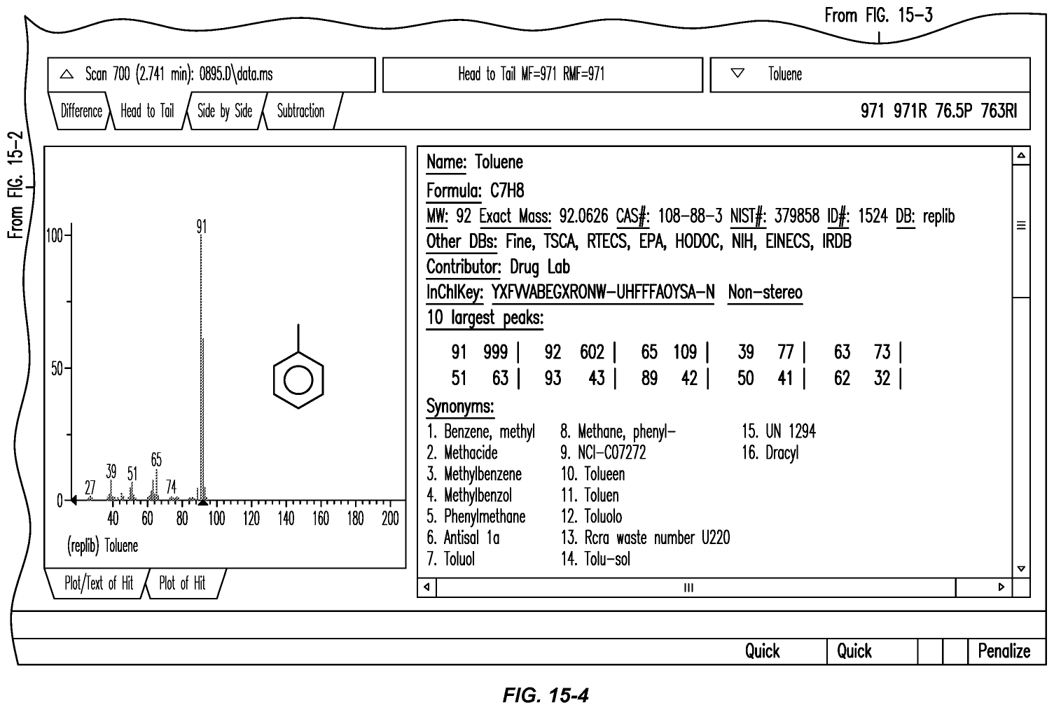

Chromatograms of the neutral taste bioBG samples, a 1,3-butylene glycol standard, and a toluene standard were compared against each other and a peak with a retention time of 2.70 min was observed in all of the samples as well as in the toluene standard. This peak was not observed in either the 1,3-butylene glycol standard or blank and was identified as toluene. This peak had the same retention time as the toluene standard peak and was a clear match to toluene through a NIST library match database search. The results for this testing can be seen in FIGS. 13-15. This compound (toluene) can be a contributor to off-flavor in a neutral odor bioBG described here.

### SPECIFIC EMBODIMENTS

Non-limiting specific embodiments are described here, each of which is considered to be within the present disclosure.

As various changes can be made in the described subject matter without departing from the scope and spirit of the present disclosure, it is intended that all subject matter contained in the description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

Embodiment 1. A 1,3-butylene glycol (BG) product comprising a bioderived BG (bioBG), wherein the BG product does not comprise one or more compounds characterized by a flavor-active region as measured by ultra-high performance liquid chromatography-quadrupole time-of-flight mass spectrometry (UHPLC/QTOF-MS), wherein the flavor-active region elutes at a retention time selected from a group consisting of: ˜5.67 mins, ˜8.202 mins, ˜8.513 mins, ˜8.758 mins, ˜9.002 mins, ˜9.435 mins, and any combinations thereof, wherein the bioBG elutes at a retention time of ˜1.005 mins.

Embodiment 2. A 1,3-butylene glycol (BG) product (e.g., liquid product, viscous, isolated) comprising a bioderived BG (bioBG), wherein the BG product does not comprise one or more compounds characterized by a flavor-active region as measured by ultra-high performance liquid chromatography-quadrupole time-of-flight mass spectrometry (UHPLC/QTOF-MS), wherein the flavor-active region comprises a relative retention time selected from a group consisting of: ˜5.614 mins, ˜8.121 mins, ˜8.429 mins, ˜8.671 mins, ˜8.913 mins, ˜9.341 mins, and any combinations thereof, where the bioBG relative retention time is 1 min.

Embodiment 3. A 1,3-butylene glycol (BG) product comprising a bioderived BG (bioBG), wherein the BG product does not comprise one or more compounds characterized by a flavor-active region as measured by ultra-high performance liquid chromatography-tandem mass spectrometry (UHPLC-MS/MS) at a collision energy of 10 V, wherein the flavor-active region elutes at a retention time selected from the group consisting of: ˜2.658 mins, ˜4.801 mins, ˜5.493 mins, ˜5.683 mins, ˜6.047 mins, ˜6.237 mins, ˜7.978 mins, ˜8.343 mins, ˜8.817 mins, ˜9.275 mins, ˜13.229 mins, and any combinations thereof, wherein the BG product comprises a bioBG elutes at a retention time of ˜0.996 min.

Embodiment 4. A 1,3-butylene glycol (BG) product comprising a bioderived BG (bioBG), wherein the BG product does not comprise one or more compounds characterized by a flavor-active region as measured by ultra-high performance liquid chromatography-tandem mass spectrometry (UHPLC-MS/MS) at a collision energy of 10 V, wherein the flavor-active region comprises a relative retention time selected from the group consisting of: ˜2.679 mins, ˜4.840 mins, ˜5.537 mins, ˜5.729 mins, ˜6.096 mins, ˜6.287 mins, ˜8.042 mins, ˜8.410 mins, ˜8.888 mins, ˜9.350 mins, ˜13.335 mins, where the relative retention time of BG is 1 min.

Embodiment 5. A 1,3-butylene glycol (BG) product comprising a bioderived BG (bioBG), wherein the BG product does not comprise one or more compounds comprising a mass spectrum as characterized by ultra-high performance liquid chromatography-tandem mass spectrometry (UHPLC-MS/MS) at a collision energy of 10 V, wherein the mass spectrum is selected from the group consisting of:
(a) 191.0737 m/z, 172.977 m/z, 141.9585 m/z, 87.0804 m/z, and 69.0700 m/z;
(b) 127.0774 m/z, 109.0650 m/z, and 81.0699 m/z;
(c) 204.1591 m/z, 159.1012 m/z, 141.9584 m/z, 89.0593 m/z, and 60.0803 m/z;
(d) 143.1070 m/z, 103.0756 m/z, 89.0599 m/z, and 71.0493 m/z;
(e) 206.1753 m/z, 143.1070 m/z, 86.0966 m/z, and 60.0807 m/z;
(f) 206.1757 m/z, 161.1178 m/z, 143.1070 m/z, 89.0601 m/z, and 71.0494 m/z;
(g) 103.0751 m/z, 89.0595 m/z, 71.0489 m/z, and 60.0804 m/z;
(h) 310.2595 m/z, 296.2439 m/z, 141.9597 m/z, 100.1129 m/z, 86.0973 m/z, and 60.0815 m/z;
(i) 310.2585 m/z, 296.2432 m/z, 161.1171 m/z, 86.0962 m/z, and 60.0805 m/z;
(j) 167.1067 m/z, 141.9587 m/z, 86.0964 m/z, and 60.0806 m/z;
(k) 199.1331 m/z, 141.9586 m/z, 100.1120 m/z, and 86.0964 m/z; and any combinations thereof, wherein the BG product comprises a bioBG mass spectrum of: 91.0756 m/z, 73.0648 m/z, and 55.0541 m/z.

Embodiment 6. The BG product according to any one of embodiments 1-5, wherein the BG product comprises BG, wherein the BG is selected from the group consisting of: (R)-1,3-BG; (S)-1,3-BG; and a mixture of (R)-1,3-BG and (S)-1,3-BG.

Embodiment 7. The BG product according to embodiment 6, wherein the BG comprises (R)-1,3-BG.

Embodiment 8. The BG product according to embodiment 6, wherein the BG comprises (S)-1,3-BG.

Embodiment 9. The BG product according to embodiment 6, wherein the BG comprises a mixture of (R)-1,3-BG and (S)-1,3-BG.

Embodiment 10. The BG product according to any one of embodiments 1-9, wherein the BG product comprises a neutral taste.

Embodiment 11. The BG product according to any one of embodiments 1-10, wherein the BG product does not comprise a biting, irritating bitterness.

Embodiment 12. A composition, comprising:
the 1,3-butylene glycol (BG) product of any one of embodiments 1-11, and
a physiologically acceptable vehicle (e.g., carrier, diluent, excipient).

Embodiment 13. The composition according to embodiment 12, further comprising an additional ingredient.

Embodiment 14. The composition according to embodiment 13, wherein the additional ingredient is selected from the group consisting of: macronutrients, such as but not limited to: proteins, carbohydrates, fats, or combinations thereof, which is used in the body of a subject to sustain growth, repair damage, aid vital processes and/or furnish energy; micronutrients, such as but not limited to, vitamins, minerals, or additional dietary ingredients; flavoring additives; dyes or color additives; and any combinations.

Embodiment 15. The composition according to any one of embodiments 12-14, wherein the BG product comprises a neutral taste.

Embodiment 16. The composition according to any one of embodiments 12-15, wherein the BG product comprises a property selected from the group consisting of: solvent, hypoglycemic agent, fungicide, bactericide, preservative, food additive, calorie source, and combinations thereof.

Embodiment 17. The composition according to any one of embodiments 12-16, wherein the BG product does not comprise compounds characterized by peaks as measured by ultra-high performance liquid chromatography-quadrupole time-of-flight mass spectrometry (UHPLC/QTOF-MS), wherein the peaks elute at a retention time of: ˜5.67 mins, ˜8.202 mins, ˜8.513 mins, ˜8.758 mins, ˜9.002 mins, and ˜9.435 mins.

Embodiment 18. The composition according to any one of embodiments 12-17, wherein the composition is a food product.

Embodiment 19. The composition according to any one of embodiments 12-18, wherein the composition is selected from the group consisting of: flavored beverages, flavored foods, high energy beverages, high energy foods, pharmaceuticals, nutraceuticals, supplements, and combinations thereof.

Embodiment 20. The composition according to any one of embodiments 12-19, wherein the composition is a high energy beverage.

Embodiment 21. The composition according to embodiment 20, wherein the BG product comprises a neutral taste bioderived (R)-1,3-BG in an amount about 1 mg/oz to about 200 mg/oz.

Embodiment 22. The composition according to any one of embodiments 12-21, wherein the BG product does not comprise a biting, irritating bitterness.

Embodiment 23. A method, comprising increasing energy (e.g., calories) in a subject in need thereof, wherein increasing energy occurs by the subject consuming a composition of any one of embodiments 12-22.

Embodiment 24. The method according to embodiment 23, wherein the composition is a food product.

Embodiment 25. The method according to embodiment 24, wherein the composition is selected from the group consisting of: flavored beverages, flavored foods, high energy beverages, high energy foods, pharmaceuticals, nutraceuticals, supplements, and combinations thereof.

Embodiment 26. Use of the 1,3-butylene glycol (BG) product of any one of embodiments 1-11 or the composition of any one of embodiments 12-22 for increasing energy (e.g., calories) in a subject in need thereof.

Embodiment 27. The use according to embodiment 22, wherein the use is of a food product.

Embodiment 28. The use according to any one of embodiments 26-27, wherein the use is of flavored beverages, flavored foods, high energy beverages, high energy foods, pharmaceuticals, nutraceuticals, supplements, or any combinations thereof.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A 1,3-butylene glycol (BG) product comprising a bioderived BG (bioBG), wherein the BG product does not comprise;
(a) one or more compounds characterized by a flavor-active region as measured by ultra-high performance liquid chromatography-quadrupole time-of-flight mass spectrometry (UHPLC/QTOF-MS), wherein the flavor-active region elutes at a retention time selected from a group consisting of: ˜5.67 mins, ˜8.202 mins, ˜8.513 mins, ˜8.758 mins, ˜9.002 mins, ˜9.435 mins, and any combinations thereof, wherein the bioBG elutes at a retention time of ˜1.005 mins;
(b) one or more compounds characterized by a flavor-active region as measured by ultra-high performance liquid chromatography-quadrupole time-of-flight mass spectrometry (UHPLC/QTOF-MS), wherein the flavor-active region comprises a relative retention time selected from a group consisting of: ˜5.614 mins, ˜8.121 mins, ˜8.429 mins, ˜8.671 mins, ˜8.913 mins, ˜9.341 mins, and any combinations thereof, where the bioBG relative retention time is 1 min;
(c) one or more compounds characterized by a flavor-active region as measured by ultra-high performance liquid chromatography-tandem mass spectrometry (UHPLC-MS/MS) at a collision energy of 10 V, wherein the flavor-active region elutes at a retention time selected from the group consisting of: ˜2.658 mins, ˜4.801 mins, ˜5.493 mins, ˜5.683 mins, ˜6.047 mins, ˜6.237 mins, ˜7.978 mins, ˜8.343 mins, ˜8.817 mins, ˜9.275 mins, ˜13.229 mins, and any combinations thereof, wherein the BG product comprises a bioBG elutes at a retention time of ˜0.996 min;
(d) one or more compounds characterized by a flavor-active region as measured by ultra-high performance liquid chromatography-tandem mass spectrometry (UHPLC-MS/MS) at a collision energy of 10 V, wherein the flavor-active region comprises a relative retention time selected from the group consisting of: ˜2.679 mins, ˜4.840 mins, ˜5.537 mins, ˜5.729 mins, ˜6.096 mins, ˜6.287 mins, ˜8.042 mins, ˜8.410 mins, ˜8.888 mins, ˜9.350 mins, 13.335 mins, wherein the relative retention time of BG is 1 min; or
(e) one or more compounds comprising a mass spectrum as characterized by ultra-high performance liquid chromatography-tandem mass spectrometry (UHPLC-MS/MS) at a collision energy of 10 V, wherein the mass spectrum is selected from the group consisting of:
(i) 191.0737 m/z, 172.977 m/z, 141.9585 m/z, 87.0804 m/z, and 69.0700 m/z;
(ii) 127.0774 m/z, 109.0650 m/z, and 81.0699 m/z;
(iii) 204.1591 m/z, 159.1012 m/z, 141.9584 m/z, 89.0593 m/z, and 60.0803 m/z;
(iv) 143.1070 m/z, 103.0756 m/z, 89.0599 m/z, and 71.0493 m/z;
(v) 206.1753 m/z, 143.1070 m/z, 86.0966 m/z, and 60.0807 m/z;
(vi) 206.1757 m/z, 161.1178 m/z, 143.1070 m/z, 89.0601 m/z, and 71.0494 m/z;
(vii) 103.0751 m/z, 89.0595 m/z, 71.0489 m/z, and 60.0804 m/z;
(viii) 310.2595 m/z, 296.2439 m/z, 141.9597 m/z, 100.1129 m/z, 86.0973 m/z, and 60.0815 m/z;
(ix) 310.2585 m/z, 296.2432 m/z, 161.1171 m/z, 86.0962 m/z, and 60.0805 m/z;
(x) 167.1067 m/z, 141.9587 m/z, 86.0964 m/z, and 60.0806 m/z;
(xi) 199.1331 m/z, 141.9586 m/z, 100.1120 m/z, and 86.0964 m/z; and any combinations thereof,
wherein the BG product comprises a bioBG mass spectrum of: 91.0756 m/z, 73.0648 m/z, and 55.0541 m/z.

2. (canceled)

3. (canceled)

4. (canceled)

5. (canceled)

6. The BG product according to claim 1, wherein the BG product comprises BG, wherein the BG is selected from the group consisting of: (R)-1,3-BG; (S)-1,3-BG; and a mixture of (R)-1,3-BG and (S)-1,3-BG.

7. The BG product according to claim 6, wherein the BG comprises (R)-1,3-BG.

8. The BG product according to claim 6, wherein the BG comprises (S)-1,3-BG.

9. The BG product according to claim 6, wherein the BG comprises a mixture of (R)-1,3-BG and (S)-1,3-BG.

10. The BG product according to claim 1, wherein the BG product comprises a neutral taste.

11. The BG product according to claim 1, wherein the BG product does not comprise a biting, irritating bitterness.

12. A composition, comprising:
the 1,3-butylene glycol (BG) product of claim 1, and
a physiologically acceptable carrier.

13. The composition according to claim 12, wherein the BG product comprises a neutral taste.

14. The composition according to claim 12, wherein the BG product comprises a property selected from the group consisting of: solvent, hypoglycemic agent, fungicide, bactericide, preservative, food additive, calorie source, and combinations thereof.

15. The composition according to claim 12, wherein the BG product does not comprise compounds characterized by peaks as measured by ultra-high performance liquid chromatography-quadrupole time-of-flight mass spectrometry (UHPLC/QTOF-MS), wherein the peaks elute at a retention time of: ˜5.67 mins, ˜8.202 mins, ˜8.513 mins, ˜8.758 mins, ˜9.002 mins, and ˜9.435 mins.

16. The composition according to claim 12, wherein the composition is a food product.

17. The composition according to claim 12, wherein the composition is selected from the group consisting of: flavored beverages, flavored foods, high energy beverages, high energy foods, pharmaceuticals, nutraceuticals, supplements, and combinations thereof.

18. The composition according to claim 12, wherein the composition is a high energy beverage.

19. The composition according to claim 18, wherein the BG product comprises a neutral taste bioderived (R)-1,3-BG in an amount about 1 mg/oz to about 200 mg/oz.

20. The composition according to claim 12, wherein the BG product does not comprise a biting, irritating bitterness.

21. The BG product of claim 1, wherein the BG product is a liquid BG product, a viscous BG product, or an isolated BG product.
